(19) United States
(12) Patent Application Publication
(10) Pub. No.: US 2004/0219551 A1
(43) Pub. Date: Nov. 4, 2004

(54) Epsti1, a novel gene induced by epithelial-stromal interaction in human breast cancer

(76) Inventors: Ole William Petersen, Hareskovby (DK); Helga Lind Nielsen, Copenhagen V (DK); Lone Ronnov-Jessen Petersen, Hareskovby (DK)

Correspondence Address: MERCHANT & GOULD PC, P.O. BOX 2903, MINNEAPOLIS, MN, 55402-0903, US

(21) Appl. No.: 10/482,978

(22) PCT Filed: Jul. 9, 2002
(86) PCT No.: PCT/DK02/00478

(30) Foreign Application Priority Data
Jul. 9, 2001 (DK) PA 2001 01074
Apr. 22, 2002 (DK) PA 2002 00601

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention describes a novel gene, EPSTI1 for epithelial stromal interaction 1 (breast), which is upregulated upon direct interaction between tumour cells and stromal cells in the tumour environment of the breast. The full-length EPSTI1 cDNA was isolated and characterized with respect to nucleotide sequence, chromosomal organization and localization, i.e. the nucleotide sequence encoding the EPSTI1 polypeptide, the EPSTI1 polypeptide itself is disclosed herein. Furthermore, the present invention discloses the use of said gene for production of pure EPSTI1-protein. Finally the use of the EPSTI1 gene as a tool or diagnosis and prognosis of cancer, especially as a specific toll to detect metastatic cancer and invasive cancer is disclosed.

### FIELD OF INVENTION

The present invention relates to a novel gene, called EPSTI1 for epithelial stromal interaction 1 (breast), the nucleotide sequence encoding the EPSTI1 polypeptide, the EPSTI1 polypeptide itself, and the use of the EPSTI1 gene as a tool for diagnosis and prognosis. The invention further relates to an expression system capable of expressing the EPSTI1 polypeptide.

### BACKGROUND

In the normal breast, the epithelial compartment is separated from the surrounding collagenous stromal tissue by an intact basement membrane. In contrast, invasive carcinoma is characterised by loss of basement membrane, and tumour cells and stromal cells are in immediate contact, which allows for direct interaction. Whereas previously assigned a passive role, the interacting stromal cells (myofibroblasts) are now considered to be critical determinants of malignancy (Elenbaas and Weinberg, 2001; Tisty and Hein, 2001). Thus, in response to epithelial malignancy, myofibroblasts have been shown to produce proteolytic enzymes directly involved in Invasion and metastasis (Examples are urokinase plasminogen activator, stromelysin-3, and matrix metalloproteinase 2 (Basset et al., 1990; Schnack Nielsen et al., 1995; Boyd and Balkwill, 1999). We have previously demonstrated that the major participating stromal cell type in the epithelial-stromal interaction in breast carcinomas is the resident fibroblast (Rønnov-Jessen et al., 1990; Rønnov-Jessen and Petersen, 1993; Rønnov-Jessen et al., 1995). Moreover, we have designed a 3-dimensional tumour environment assay, which allows critical aspects of tumour histology to be recapitulated in culture (Rønnov-Jessen et al., 1992; Rønnov-Jessen et al., 1995).

There is mounting evidence in support of the view that malignancy results from the interaction of tumour cells and the surrounding stroma, in particular the myofibroblasts (Liotta and Kohn, 2001; Radisky et al., 2001; Tisty and Hein, 2001). So far only a few genes directly involved in this process have been identified. One example is stromelysin-3, which was originally reported to be overexpressed in the stroma of breast carcinomas (Basset et al., 1990). Further studies have broadened the significance of stromelysin-3 expression to Include tumours of other tissues (Basset et al., 1993) and stromelysin-3 has now been established as an independent prognostic marker of malignancy (Engel et al., 1994; Ahmad et al., 1998). However, although prognostic markers can be used to design improved cancer treatment strategies and thus improve the life-quality of the individual cancer patient, an even more important aspect is to identify new diagnostic markers which may improve the survival of the patients via an earlier and more accurate diagnosis. Thus there is a call for the identification of more accurate diagnostic as well as prognostic markers.

WO 99/38881 discloses a range of nucleotide sequence of which gene no. 64 encodes a protein thought to be important in cytoskeletal regulation and targeting. Gene no. 64 is believed to reside on chromosome 13 and is expressed primarily in human adult small intestine and ovarian tumour tissue, and to a lesser extent In T cells, lymphoma tissue and dendritic cells. The polynucleotides and polypeptides are described as useful as reagents for differential identification of the described tissues and cell types and furthermore for diagnosis of diseases such as gastrointestinal, immune or reproductive disorders, and in particular proliferative disorders, particularly of the digestive tract.

WO 00/11014 discloses a range of nucleotide sequence and encoded polypeptides of which gene no. 23 (SEQ ID NO 33) encodes SEQ ID NO 151 which is described as an polypeptide with a transmembrane domain. This polypeptide is believed to share structural features to type Ia membrane proteins. The polynucleotides and polypeptides are suggested as being useful as reagents for differential identification of tissues or cell types and for diagnosis of diseases and conditions such as immune or hematopoietic diseases and/or disorders, particularly inflammatory conditions or immunodeficiencies such as AIDS.

Since the tumour cells and the surrounding stroma play a key role in the development of cancer, the identification of genes, the expression of which is directed by this interaction provides a novel avenue for identifying genes which are likely to have important implications for future strategies of treatment of cancer.

### SUMMARY OF INVENTION

It is a significant objective of the present invention to identify a gene which is regulated by the interaction between tumour cells and the surrounding stroma cells and consequently may prove to be useful in cancer therapy, diagnosis and/or prognosis.

The present invention describes a novel gene, EPSTI1 for epithelial stromal interaction 1, which is upregulated upon direct interaction between tumour cells and stromal cells in the tumour environment of the breast. The full-length EPSTIL cDNA was isolated and characterised with respect to nucleotide sequence, chromosomal organisation and localisation. Furthermore, the present invention discloses the use of said gene for production of pure EPSTI1-protein. Finally the use of the EPSTI1 gene as a tool for diagnosis and prognosis is disclosed.

### DETAILED DESCRIPTION

During growth, invasion and metastasis, tumour cells interact extensively with the surrounding stroma. To identify genes which are switched on or off during this process, a previously described tumour environment assay was used.

Using a 3-dimensional tumour environment assay, which recapitulates critical aspects of the microenvironment in vivo, including typical tissue histology, a novel human gene, designated EPSTI1, was identified by differential display (example 1). To isolate this gene, the profiles of mRNA pooled from tumour cells (MCF-7) and fibroblasts (human telomerase (hTERT) transduced normal breast fibroblasts, D533) cultured in separate cultures were compared to the mRNA profile of the cells cultured together and analysed by differential display. The isolated transcripts were sequenced, and two of the amplicons represented a hitherto unknown human gene, which was upregulated in epithelial-stromal co-cultures. By normalising RNA ratios using lineage-specific markers including vimentin and cytokeratin 19, differential expression was confirmed by real time-PCR. A full-length cDNA of 1508 bp was generated by 5′ rapid amplification of cDNA ends and included an open reading frame encoding a 307 aa protein, the EPSTI1 polypeptide. The EPSTI1 polypeptide has an molecular mass of 35 kDa to 45 kDa, such as in the range from 35-45 kDa, e.g. in the range from 37-45, e.g. from 38-44, e.g. from 39-44, e.g. 39-43, such as in the range from 39-42, e.g. from 40-42.

The novel gene, EPSTI1, is mapped to the long arm of chromosome 13, example 2, FIG. 5.

The gene was initially designated BRESI-1 for breast epithelial-stromal interaction-1 gene (in the Danish patent appl. PA 2001 01074). However, by suggestion of the Gene Nomenclature Committee (HGNC) of the Human Genome Organisation (HUGO) the gene was renamed to EPSTI1 for Epithelial Stromal Interaction 1 (breast). The EPSTI1 name was approved by the HGNC on August 31st, 2001 and represents a new root symbol. In contrast to the previously suggested gene symbol BRESIL (breast epithelial-stromal interaction-1), this nomenclature avoids any strong reference to tissue specificity. The EPSTI1 (BRESI-1) sequence appears under the GenBank accession no. AF396928. EPSTI1 shared no sequence identity to any gene with a recognised function within the BLASTN database. More recently (Jan 22nd, 2002) similar sequences have been Isolated from a primary mammary tumour and a mammary tumour metastasized to lung in the mouse (Mus musculus). At the nucleotide level the mouse sequences (Genbank accession no.s BC021821 and BC020120 in the NCBI database) display identity in 559 out of 661 aligned nucleotides to EPSTI1. Also, a transcript with similarity to EPSTIL has subsequently been described in the rhesus monkey with B-cell non-Hodgkins lymphoma (Macaca mulatta, NCBI accession no. AJ414515, identity in 175 out of 182 nucleotides). Finally, expressed sequence tags representing EPSTI1 have been described in 11 SAGE (serial analysis of gene expression) libraries, which include normal mammary gland epithelium, human microvascular endothelial cells, primary ovary carcinoma, colon adenocarcinoma, gastic carcinoma and neoplastic pancreas (NCBI Sage gene to tag mapping, Unigene cluster Id: Hs 343800). Several EST clones did align to EPSTI1, and the sequence was initially mapped in silico to human chromosome 13q14.2 (example 1, FIG. 3), and after the recent annotation of the NCBI database, EPSTI1 was mapped to 13q13.3 (example 2, FIG. 5). The position on chromosome 13 q was confirmed by PCR on human monochromosomal hybrids (Drwinga et al. (1993) Genomics 16: 311-314.) covering fragments of chromosome 13 (example 2, FIG. 5). The deduced protein sequence shows 64% overall Identity to a putative mouse protein (NBCI accession no. BAB30623).

Real time PCR reveals that the expression of EPSTI1 was predominantly restricted to thymus, stomach, lung, small intestine, spleen, prostate, adrenal gland, pancreas, liver, uterus, salivary gland, testis and placenta with the highest level of expression in the latter. Most importantly, however, an interesting attribute of this gene is that it is overexpressed (up to 122 times) in primary breast carcinomas (example 1 and 5) and up to 158 times in metastases as compared to normal breast. The fact that placenta can be considered to harbour extensive Invasion, albeit as a perfectly controlled process, and the fact that EPSTI1 was pulled out from a model of direct interaction of tumour cells and myofibroblasts, lead to the hypothesis that the discovery of EPSTI1 has implications for future strategies of diagnosis, prognosis and treatment of cancer in particular. Since cancer invasion is the ultimate outcome of tumour cell-fibroblast interaction (Liotta and Kohn, 2001), identification and characterisation of genes such as EPSTI1 expressed In the tumour environment are markers of invasion and metastasis or even a valuable tool in prenatal diagnostics.

The present application thus describes an isolated nucleic acid molecule encoding the polypeptide EPSTI1 (SEQ ID NO:2) or polypeptides having a homology of at least 70%, such as at least 74%, to the polypeptide with a amino acid sequence as shown in FIG. 2B (SEQ ID NO:2) of mammalian origin, preferably human origin.

The nucleic acid of the application can further be described as an isolated nucleic acid molecule encoding a polypeptide selected from the group consisting of:

a) the polypeptide EPSTI1 set forth in SEQ ID NO:2;

b) a polypeptide having a homology of at least 70% to the polypeptide sequence of SEQ ID NO:2;

c) a fragment of the polypeptide defined in a) or b) of at least 9 amino acids; and

d) a polypeptide comprising a fragment of SEQ ID NO: 2 comprising at least 9 consecutive amino acids of SEQ ID NO: 34;

e) the nucleic acid sequence of SEQ ID NO:1 encoding the EPSTI1 polypeptide;

f) a nucleic acid having a homology of at least 90% to the nucleic acid sequence of SEQ ID NO: 1; and

g) a nucleic acid sequence which hybridises under stringent conditions to the protein coding regions of SEQ ID NO: 1 encoding the polypeptide of SEQ ID NO: 34.

As commonly defined (se e.g. Encyclopaedia of Life Sciences/www.els.net, Nature Publishing Group, 2000) “homology” is here defined as sequence identity between genes or proteins at the nucleotide or amino acid level, respectively. Thus, in the present context “sequence identity” is a measure of identity between proteins at the amino acid level and a measure of identity between nucleic acids at nucleotide level. The protein sequence Identity may be determined by comparing the amino acid sequence in a given position in each sequence when the sequences are aligned. Similarly, the nucleic acid sequence Identity may be determined by comparing the nucleotide sequence in a given position in each sequence when the sequences are aligned

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % Identity # of identical positions/total # of positions (e.g., overlapping positions)×100). In one embodiment the two sequences are the same length.

One may manually align the sequences and count the number of identical amino acids. Alternatively, alignment of two sequences for the determination of percent identity can be accomplished using a mathematical algorithm. A preferred, non-limiting example of a mathematical algorithm utilised for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12, to obtain nucleotide sequences homologous to a nucleic acid molecules of the invention. BLAST protein searches can be performed with the XBLAST program, score=50, wordlength=3 to obtain amino acid sequences homologous to a protein molecule of the invention. To obtain gapped alignments for comparison purposes, Gapped BLAST can be utilised as described in Altschul et al. (1997) Nucleic Acids Res. 25:3389-402. Alternatively, PSI-Blast can be used to perform an Iterated search which detects distant relationships between molecules. When utilising the NBLAST, XBLAST, and Gapped BLAST programs, the default parameters of the respective programs can be used. See http://www.ncbi.nlm.nih.gov. Alternatively, sequence identity can be calculated after the sequences have been aligned e.g. by the program of Pearson W. R and D. J. Lipman (Proc Natl Acad Sci USA 85:2444-2448, 1998) in the EMBL database (www.ncbi.nim.gov/cgi-bin/BLAST). Generally, the default settings with respect to e.g. “scoring matrix” and “gap penalty” can be used for alignment. In the present application the BLASTN and PSI BLAST default settings was used.

The percent identity between two sequences can be determined using techniques similar to those described above, with or without allowing gaps. In calculating percent identity, only exact matches are counted.

The concept of “polypeptide similarity” takes the concept of conservative amino acid substitutions into account. The term “conservative substitutions” as used herein denotes the replacement of an amino acid residue by another, biologically similar residue. Examples of conservative substitutions include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like. The term “conservative substitutions” also includes the use of a substituted amino acid in place of an unsubstituted parent amino acid provided that antibodies raised to the substituted polypeptide also immunoreact with the unsubstituted polypeptide. Thus, in the present context, the term “polypeptide similarity” is a measure of similarity between two optimally aligned amino acid sequences in which both identical as well as amino acids which qualify as conservative substitutions are counted.

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptide may be identical to the coding sequence shown in FIG. 2A (SEQ ID NO: 1) or may be a different coding sequence which coding sequence, as a result of the redundancy or degeneracy of the genetic code, encodes the same mature polypeptide as of FIG. 2B (SEQ ID NO:2) or significant fragments, analogues and derivatives of the polypeptide. It is thus understood that a nucleic acid sequence which is complementary to any of the nucleic acid sequences described herein is covered by the present application.

The polynucleotide of the present invention is thus furthermore characterised as a nucleic acid sequence which hybridises under stringent conditions to the nucleic acid sequence defined above (SEQ ID NO:1) or fragments thereof comprising at least 15 nucleic acids, e.g. a nucleic acid sequence hybridising to the protein coding regions of SEQ ID NO: 1 or fragments thereof and which comprises at least 15 nucleic acids.

The term “stringent conditions” when used in conjunction with hybridisation conditions is as defined in the art, i.e. 15-20° C. under the melting point Tₘ, cf. Sambrook et al, 1989, pages 11.45-11.49. Preferably, the conditions are “highly stringent”, i.e. 5-10° C. under the melting point Tₘ.

Polynucleotides according to the present invention are also nucleic acids having a homology of at least 89% to the nucleic acid sequence of SEQ ID NO: 1 such as 89%, e.g. 90%. 91%, 92%, 93%, e.g. 94%, 95%, 96%, 97%, 98%, such as 99% and which comprises at least 15 nucleic acids. In the present application “homology” of nucleic acid sequences is defined as sequence identity between the nucleic acid sequences and it may be determined by comparing a position in each sequence which is aligned as described previously for purposes of comparison. When a position in the compared sequence is occupied by the same nucleotide base, then the molecules are identical or homologous at that position.

The polynucleotide which encodes the mature polypeptide of FIG. 2B (SEQ ID NO:2) may include, but is not limited to only the coding sequence for the mature polypeptide; the coding sequence for the mature polypeptide and additional coding sequence such as a leader or secretory sequence or a pro-protein sequence; the coding sequence for the mature polypeptide (and optionally additional coding sequence) and non-coding sequence, such as introns or non-coding sequence 5′ and/or 3′ of the coding sequence for the mature polypeptide are also included.

The present invention further relates to variants of the hereinabove described polynucleotides which encode significant fragments, analogues and derivatives of the polypeptide having a homology of at least 70% such as 74%, e.g. 75%, 76%, 77%, 78%, 79%, such as 80% homology, e.g. 81%, 8²%, 83%, 84%, ⁸5%, 86%, 87%, 88%, ⁸9%, such as 90% homology, e.g. 91%, 92%, 93%, 94%, 95%, 96%, 97%, e.g. at least 98% homology, such as 99% homology to the polypeptide sequence in FIG. 2B (SEQ ID NO:2).

The variant of the polynucleotide may be a naturally occurring allelic variant of the polynucleotide or a non-naturally occurring variant of the polynucleotide; or a polynucleotide encoding a polypeptide of at least 9 consecutive amino acids having a homology of at least 70%, such as 74%, e.g. 75%, 76%, 77%, 78%, 79%, such as 80% homology, e.g. 81%, 82%, 83%, 84%, 85%, 86%, 87%, 88%, 89%, such as 90% homology, e.g. 91%, 92%, 93%, 94%, 95%, 96%, 97%, e.g. 98% homology, such as 99% homology to the polypeptide sequence of SEQ ID NO:34.

Thus, the present invention includes polynucleotides encoding the same mature polypeptide as shown in FIG. 2B (SEQ ID NO:2) as well as variants of such polynucleotides which variants encode for a fragment, derivative or analogue of the polypeptide of FIG. 2B (SEQ ID NO:2). Such nucleotide variants include deletion variants, substitution variants, addition variants and insertion variants.

As hereinabove indicated, the polynucleotide may have a coding sequence which is a naturally occurring allelic variant of the coding sequence shown in FIG. 2A (SEQ ID NO: 1).

As known in the art, an allelic variant is an alternate form of a polynucleotide sequence which may have a substitution, deletion or addition of one or more nucleotides, which does not substantially alter the function of the encoded polypeptide.

The polynucleotides of the present invention may also have the coding sequence fused in frame to a marker sequence which allows for purification of the polypeptide of the present invention. The marker sequence may be a hexa-histidine tag supplied by a pQE-9 vector to provide for purification of the mature polypeptide fused to the marker in the case of a bacterial host, or, for example, the marker sequence may be a hemagglutinin (HA) tag when a mammalian host, e.g. COS-7 cells, is used. Non-limiting examples of additional fusion polypeptides comprising at least one polypeptide fragment according to the present Invention and at least one fusion partner are fusion proteins wherein said fusion partner is selected from the group consisting of green fluorescent protein (GFP), glutathione-S-transferase (GST), Maltose-binding protein (MBP), an epitope tag comprising the Myc proto-oncogene, bacterial thioredoxin, FLAG epitope (Asp-Tyr-Lys-Asp-Asp-Asp-Asp-Lys) and viral V5 epitope.

The polynucleotides of the present invention may further comprise heterologous nucleotide sequences and even further comprise heterologous polypeptides encoded by said nucleotide sequences.

The term a “heterologous nucleotide sequence” is herein used to describe a DNA sequence Inserted within or connected to another DNA sequence which codes for polypeptides not coded for in nature by the DNA sequence to which it is joined. Allelic variations or naturally occurring mutational events do not give rise to a heterologous DNA sequence as defined herein.

The term a “heterologous polypeptide” is herein used to describe a polypeptide that is being expressed in a context not found in nature.

Fragments of the EPSTI1 gene may be used as a hybridisation probe for a cDNA library to isolate the full length EPSTI1 gene and to isolate other genes which have a high sequence similarity to the EPSTI1 gene or similar biological activity both from human as well as other sources. Probes of this type preferably have at least 30 bases and may contain, for example, 50 or more bases. The probe may also be used to identify a cDNA clone corresponding to a full-length transcript and a genomic clone or clones that contain the complete gene including regulatory and promotor regions, exons, and introns. An example of a screen comprises isolating the coding region of the gene by using the known DNA sequence to synthesise an oligonucleotide probe. Labelled oligonucleotides having a sequence complementary to that of the gene of the present invention are used to screen a library of human cDNA, genomic DNA or mRNA to determine which members of the library the probe hybridises to.

The term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

The present invention further relates to polynucleotides, which hybridise to the hereinabove-described sequences if there is at least 70%, e.g. at least 88%, e.g. 89%, e.g. 90%, such as 91%, 92%, 93%, 94%, such as 95% identity, e.g. 96%, 97%, 98% such as 99% identity between the sequences. The present invention particularly relates to polynucleotides, which hybridise under stringent conditions to the hereinabove-described polynucleotides. The polynucleotides which hybridise to the hereinabove described polynucleotides in a preferred embodiment encode polypeptides which either retain substantially the same biological function and/or activity as the mature polypeptide encoded by the cDNAs of FIG. 2A. (SEQ ID NO: 1).

Alternatively, the polynucleotide may have at least 17 bases, such as 17 bases, e.g. 18 bases, e.g. 19 bases, e.g. 20 bases, such as 22 bases, e.g. 23, 24, 25, 26, 27, 28, 29, such as 30 bases, e.g. 31 bases, e.g. 32 bases, e.g. 33 bases, e.g. 34 bases, e.g. 35 bases, e.g. 36 bases, e.g. 37 bases, such as 38 bases, e.g. 39 bases for example at least 50 bases which hybridise to a polynucleotide of the present invention and which has an identity thereto, as hereinabove described, and which may or may not retain activity. For example, such polynucleotides may be employed as probes for the polynucleotide of SEQ ID NO:1, for example, for recovery of the polynucleotide or as a diagnostic probe or as a PCR primer.

Thus, the present invention is directed to polynucleotides having at least a 70% Identity, e.g. 75%, such as 80%, e.g. 87%, 88%, 89%, such as 90%, such as 90%, 91%, 92%, 93%, 94%, 95%, 96%, 97%, 98%, such as 99% identity to a polynucleotide which encodes the polypeptide of SEQ ID NO:2 as well as fragments thereof, which fragments have at least 30 bases and preferably at least 50 bases and to polypeptides encoded by such polynucleotides.

Furthermore, the invention relates to an oligonucleotide capable of hybridising to a nucleic acid of SEQ ID NO: 1 for use as a medicament, i.e. the employment of antisense technology to down regulate or even Inhibit the transcription and/or translation process of the EPSTI1 gene into its polypeptide product, thereby being able to control the level of EPSTI1 protein in tissue culture or an organism, e.g. in a mammalian such as a human.

The present invention further relates to a polypeptide which has the deduced amino acid sequence of FIG. 2B (SEQ ID NO:2), as well as fragments, analogues and derivatives of such polypeptide. The isolated polypeptide comprises an amino acid sequence selected from the group consisting of:

a) the polypeptide EPSTI1 set forth in SEQ ID NO:2;

b) a polypeptide having a homology of at least 70% to the polypeptide sequence of SEQ ID NO:2;

c) a fragment of the polypeptide defined in a) or b) of at least 9 amino acids; and

d) a polypeptide comprising a fragment of SEQ ID NO: 2 comprising at least 9 consecutive amino acids of SEQ ID NO: 34.

The polypeptide may be a substantially purified polypeptide. In the present context, the term “substantially purified” or “substantially pure” is understood to mean that the polypeptide in question is substantially free from other components, e.g. other polypeptides or carbohydrates, which may result from the production and/or recovery of the polypeptide or otherwise be found together with the polypeptide. The purity of a protein may be assessed by SDS gel electrophoresis, and in the present context a preparation of substantially pure polypeptide only show 1 component on a Coomassie coloured SDS gel.

The terms “fragment”, “derivative” and “analogue” when referring to the polypeptide of FIG. 2B (SEQ ID NO:2) means a polypeptide which retains essentially the same biological function or activity as such polypeptide. Thus, an analogue includes a pro-protein which can be activated by cleavage of the pro-protein portion to produce an active mature polypeptide. The term “fragment” as used herein further refers to an amino acid sequence comprising a subsequence of a peptide of the invention. Said fragment is a peptide having one or more immunogenic determinants of the EPSTI1 protein. Fragments can inter alia be produced by enzymatic cleavage of precursor molecules, using restriction endonucleases for the DNA and proteases for the polypeptides. Other methods include chemical synthesis of the fragments or the expression of peptide fragments by DNA fragments. The polypeptide of the present invention may be a recombinant polypeptide, a natural polypeptide or a synthetic polypeptide, preferably a recombinant polypeptide.

The fragment, derivative or analogue of the polypeptide of FIG. 2B (SEQ ID NO:2) may be (i) one in which one or more of the amino acid residues are substituted with a conserved or non-conserved amino acid residue (preferably a conserved amino acid residue) and such substituted amino acid residue may or may not be one encoded by the genetic code, i.e. the polypeptide or polypeptide fragment may be modified by conservative substitutions, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a pro-protein sequence. Such fragments, derivatives and analogues are deemed to be within the scope of those skilled in the art from the teachings herein. Accordingly, the fragment, derivative or analogue of the polypeptide of FIG. 2B may be coupled to a carbohydrate or a lipid moiety and it may be glycosylated and/or phospho-rylated.

The polypeptides and polynucleotides of the present invention are preferably provided in an isolated form, and preferably are purified to homogeneity.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The polypeptides of the present invention include the polypeptide of SEQ ID NO:2 as well as polypeptides which have at least 70% similarity (preferably at least a 70% homology) to the polypeptide of SEQ ID NO:2 and polypeptides having at least a 90% similarity (more preferably at least a 90% homology) to the polypeptide of SEQ ID NO:2 and polypeptides having at least a 95% similarity (still more preferably a 95% homology) to the polypeptide of SEQ ID NO:2 and also include portions of such polypeptides with such portion of the polypeptide generally containing at least 9 amino acids, such as 10 amino acids, such as 11, 12, 13, 14, 15, 16, 17, 18, 19, 20, such as 25, such as 30 amino acids and more preferably at least 50 amino acids.

Furthermore, the polypeptide of the present invention may be a polypeptide having a homology of at least 70% such as 74%, e.g. 75%, 76%, 77%, 78%, 79%, such as 80% homology, e.g. 81%, 82%, 83%, 84%, 85%, 86%, 87%, 88%, 89%, such as 90% homology, e.g. 91%, 92%, 93%, 94%, 95%, 96%, 97%, e.g. at least 98% homology, such as 99% homology to the polypeptide sequence In FIG. 2B (SEQ ID NO:2).

Polypeptide homology is typically analysed using sequence analysis software such as the Sequence Analysis Software Package of the Genetics Computer Group, University of Wisconsin Biotechnology Center, Madison, Wis.). Polypeptide sequence analysis software matches homologous sequences using measures of homology assigned to various substitutions, deletions, substitutions, and other modifications.

The substantially pure EPSTI1 polypeptide may be used as a medicament.

The present invention also relates to vectors which include polynucleotides of the present invention, host cells that are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The polynucleotides of the present invention may be employed for producing polypeptides according to the invention by recombinant techniques comprising

(a) culturing a host cell under conditions suitable to produce a polypeptide encoded by the nucleic acid molecule of; and

(b) recovering the polypeptide from the cell culture.

Thus, for example, the polynucleotide may be included in any one of a variety of expression vectors for expressing a polypeptide. Such vectors include chromosomal, non-chromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known In the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda PL promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin-derivatives (G418, geneticin) resistance for eukaryotic cell culture, or such as tetracycline or ampicillin resistance in E. coli and other bacteria.

The vector containing the appropriate DNA sequence as hereinabove described, as well as an appropriate promoter or control sequence, may be employed to transform an appropriate host to permit the host to express the protein.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; MCF7 breast cancer cells; MCF-10A cells; MCF-7 S9 cells; HMT-3909 cells; HMT-3522 cells; T47D cells; ZR-75 cells; BT-20 cells; MDA-MB-435 cells; HeLa cells; plant cells, etc. The selection of an appropriate host is deemed to be within the scope of those skilled in the art from the teachings herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen c/o Merck, Albertslund, Denmark)), pBS, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, PMSG, PSVL (Pharmacia). However, any other plasmid or vector may be used as long as they are replicable and viable In the host.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, lipofection, electroporation or any of a number of other transfection methods known to the skilled artesian. The EPSTI1 coding DNA may also be inserted into the host cell by transduction with an appropriate engineered virus particle. Examples of useful virus systems comprise retrovirus, adenovirus and adeno-associated virus. Many useful retroviral vectors are based on murine retroviruses. They can carry 6 to 7 kb of foreign DNA (promoter+cDNA) but suffer from the draw-backs of requiring the development of high titer packaging lines, requiring that target cells is dividing, and are subject to host cell down-modulation. Adenoviral vectors can be produced at high levels and do not require a dividing target cell, but they do not normally integrate, resulting in only transient expression. Adeno-associated viral vectors are defective parvoviruses that integrate Into a non-dividing host cell at a specific location (19 q). Disadvantages are genetic instability, small range of insert size (2-4.5 kb), and thus far, only transient expression.

The constructs in host cells can be used in a conventional manner to produce the gene product encoded by the recombinant sequence. Alternatively, the polypeptides of the invention can be synthetically produced by conventional peptide synthesisers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples include the SV40 enhancer on the late side of the replication origin bp 100 to 270, a cytomegalovirus early promoter enhancer, the polyoma enhancer on the late side of the replication origin, and adenovirus enhancers.

The polypeptide can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

The polynucleotides and polypeptides of the present Invention may be employed as research reagents and materials for discovery of treatments and diagnostics to human disease.

The polypeptides, their fragments or other derivatives, or analogues thereof, or cells expressing them can be used as an immunogen to produce antibodies thereto. These antibodies can be, for example, polyclonal or monoclonal antibodies. The present invention also includes chimera, single chain, and humanised antibodies, as well as Fab fragments, or the product of an Fab expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a non-human. The antibody so obtained will then bind the polypeptides itself. In this manner, even a sequence encoding only a fragment of the polypeptides can be used to generate antibodies binding the whole native polypeptides. In the present application a “significant fragment” of the polypeptide according to the Invention is a polypeptide fragment of at least 9 amino acids capable of generating antibodies useful for detecting EPSTI1 polypeptides. Such antibodies can then be used to isolate the polypeptide from tissue expressing said polypeptide.

For preparation of monoclonal antibodies, any technique which provides antibodies produced by continuous cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanised antibodies to immunogenic polypeptide products of this invention.

This invention is also related to the use of the gene of the present Invention as a tool for diagnostics. Detection of increased levels of EPSTI1 transcripts (mRNA) or EPSTI1 polypeptide or detection of a mutated form of EPSTI1 will allow a diagnosis of a disease or a susceptibility to a disease, for example, related to cancer, such as invasive cancer and/or metastatic cancer.

Individuals having elevated levels of mRNA transcripts or having mutations of the gene of the present invention may be detected at the nucleic acid level by a variety of techniques. Nucleic acids for diagnosis may be obtained as samples from a patient, e.g. from the patient's tissue, body fluids or cells.

Thus, the present invention covers a method for determining the presence of EPSTI1 mRNA in a sample, the method comprising:

a) obtaining a sample comprising mRNA from a test subject;

b) contacting the test sample with an isolated nucleic acid molecule that hybridizes under conditions of hybridisation to the EPSTI1 mRNA; and

c) determining that the EPSTI1 mRNA is present in the sample when the sample contains mRNA that selectively hybridises to the isolated nucleic acid molecule;

wherein the EPSTI1 mRNA is selected from the group consisting of:

d) a mRNA molecule that encodes a polypeptide comprising the amino acid sequence of SEQ ID NO:2;

e) a mRNA molecule corresponding to the nucleic acid sequence SEQ ID NO: 1; or the complement thereof;

f) a mRNA molecule which comprises at least 9 contiguous nucleotides selected from the nucleic acid sequence SEQ ID NO: 1.

In the present context the term “test subject” refers to the cell, cell culture, tissue, organism or sample of said organism to be tested. Furthermore, the expression “conditions of hybridisation” refer to conditions, which allow two complementary, or partially complementary nucleic acid molecules to associate into a hybrid molecule by base pairing. Typical conditions of hybridisation can be found in the art, e.g. in Ausubel et al. (2000) and in Sambrook et al, (1989), both of which are incorporated by reference.

Similarly, the present invention also describes a method for determining the relative level of EPSTI1 mRNA in a sample, the method comprising:

a) obtaining a sample comprising mRNA from a test subject and from a control subject;

b) contacting the test sample the control sample with at least one nucleic acid molecule that hybridizes under conditions of hybridisation to the EPSTIL mRNA; and

c) determining the realtive level of the EPSTI1 mRNA In the test sample by comparing the EPSTIL mRNA specific signal In the test sample to the signal in the control sample.

wherein the EPSTIL mRNA is selected from the group consisting of:

d) a mRNA molecule that encodes a polypeptide comprising the amino acid sequence of SEQ ID NO:2;

e) a mRNA molecule corresponding to the nucleic acid sequence SEQ ID NO:1; or the complement thereof;

f) a mRNA molecule which comprises at least 9 contiguous nucleotides selected from the nucleic acid sequence SEQ ID NO:1.

In the present context the term “control subject” refer a cell, a cell culture, a tissue sample, an organism or sample of said organism characterised by containing a previously determined level of EPSTI1 mRNA.

Such determination of the relative level of specific mRNA's in a sample can be performed by one of several methods well known to those skilled in the art as described in Sambrook, et al. (1989) Molecular Cloning: A Laboratory Manual, Second Edition, Cold Spring Harbor, N.Y., and Ausubel et al. (2000) Current protocols in molecular biology, John Wiley and Sons, Inc. The disclosure of both are hereby incorporated by reference. One particular convenient method of determining the relative level of EPSTI1 mRNA is by real-time PCR using primers that are specific for various mRNAs as described in example 1, 5 and 6. However the relative level of EPSTI1 mRNA may also be determined by northern blots (example 3) provided that the blot comprises a control sample and that the blot in addition to an EPSTI1-probe (eg. the probe described in example 3) also is probed with one or more control probes such as a probe specific for GAPDH, TBP, AGP or ribosomal RNA.

The diagnostic method may be performed on a sample comprising an extract from a cancer tissue or a suspected cancer tissue, wherein the sample primarily is isolated from tissues selected from the group of tissues consisting of breast, placenta, ovary, testis, thymus, lymphoid tissue, lung, stomach, small intestine, colon, pancreas, stomach, spleen, skin and extracellular body fluids, however other tissues may be considered as well.

By the term “sample” is meant the material suspected of containing the nucleic acid or protein to be studied. Such samples include biological fluids such as blood, serum, plasma, sputum, lymphatic fluid, semen, vaginal mucus, faeces, urine, spinal fluid, and the like; biological tissue such as hair and skin; and so forth. Even if the EPSTI1 protein is not a secreted protein, it may bind to other proteins, glycolipids, vesicles or the like, which may render it secretable and thus measurable in biological fluids. When necessary, the sample may be pre-treated with reagents to liquefy the sample and release the nucleic acids from binding substances. Such pre-treatments are well known in the art.

In the present context, the term “extracellular body fluids” describes the extracellular fluids of the mammalian organism. Examples are: blood, ascites, plasma, lymph, amnion fluid, and cerebrospinal fluid.

The nucleic acid may be used directly for detection or may be amplified enzymatically by using PCR prior to analysis. RNA or cDNA may also be used for the same purpose. As an example, PCR primers complementary to the nucleic acid encoding EPSTIL can be used to identify and analyse the expression level or mutations. Furthermore, deletions and insertions can be detected by direct sequencing or sequencing of PCR products or as a change in size of the amplified product in comparison to the normal genotype. Point mutations can be identified by hybridizing amplified DNA to radiolabelled EPSTI1 RNA or alternatively, radiolabelled EPSTI1 antisense DNA sequences. Perfectly matched sequences can be distinguished from mismatched duplexes by RNase A digestion or by differences In melting temperatures.

Sequence differences between the reference gene and genes having mutations may be revealed by the direct DNA sequencing method. In addition, cloned DNA segments may be employed as probes to detect specific DNA segments. The sensitivity of this method is greatly enhanced when combined with PCR. For example, a sequencing primer is used with double-stranded PCR product or a single-stranded template molecule generated by a modified PCR. The sequence determination is performed by conventional procedures with radiolabelled nucleotide or by automatic sequencing procedures with fluorescent-tags.

In addition to more conventional gel-electrophoresis and DNA sequencing, mutations can also be detected by in situ analysis.

As described above, the present invention also relates to a diagnostic assay for detecting altered levels of EPSTIL mRNA and/or protein in various tissues since an over-expression of the proteins compared to normal control tissue samples can detect the presence of cancer such as metastatic cancer and/or invasive cancer or give a prognostic indication of the risk of developing cancer, such as metastatic cancer and/or invasive cancer. A high level of this protein Is indicative of an invasive cancer, since it has herein been shown that invasive breast carcinomas cells have increased levels of EPSTI1. As shown In the examples, the EPSTI1 is upregulated during direct epithelial-stromal Interaction as shown by the expression level In normal breast versus invasive breast carcinomas.

Assays used to detect levels of EPSTI1 protein in a sample derived from a host are well-known to those of skill in the art and include radioimmunoassays, competitive-binding assays, Western Blot analysis and preferably an ELISA assay. An ELISA assay initially comprises preparing an antibody specific to the EPSTI1 antigen, such as a polyclonal antibody, preferably a monoclonal antibody. In addition a reporter antibody is prepared against the monoclonal antibody. To the reporter antibody is attached a detectable reagent such as radioactivity, fluorescence or in this example a horseradish peroxidase enzyme. A sample is now removed from a host and incubated on a solid support, e.g. a polystyrene dish, that binds the proteins in the sample. Any free protein binding sites on the dish are then covered by incubating with a non-specific protein such as bovine serum albumin. Next, the monoclonal antibody is incubated in the dish during which time the monoclonal antibodies attach to any EPSTI1 proteins attached to the polystyrene dish. All unbound monoclonal antibody is washed out with buffer. The reporter antibody linked to horseradish peroxidase is now placed In the dish resulting in binding of the reporter antibody to any monoclonal antibody bound to EPSTI1. Unattached reporter antibody is then washed out. Peroxidase substrates are then added to the dish and the amount of colour developed in a given time period is a measurement of the amount of EPSTI1 protein present in a given volume of patient sample when compared against a standard curve.

Thus the present application describes a method for determining the presence of a EPSTI1 protein in a sample comprising the steps:

a) contacting a sample or preparation thereof with an antibody or antibody fragment according to the invention which selectively binds the EPSTI1 polypeptide; and

b) detecting whether said EPSTI1 polypeptide is bound by said antibody and thereby detecting the EPSTI1 polypeptide.

The antibodies may be labelled. The label may be selected from the group consisting of radioisotopes, fluorescent compounds, enzymes, chemoluminescent compounds or a member of an affinity pair.

As used herein, the expression “affinity pair” describes two molecules which specifically associates with each other given the proper conditions, one example of an affinity pair is the bioitin-avidin affinity pair. Representative examples of affinity pairs are given in table 4.

TABLE 4
Representative Specific Affinity Pairs

| antigen | Antibody |
|---|---|
| hapten (e.g. digoxigenin) | anti-hapten antibody or Fab-fragment |
| biotin | avidin (or streptavidin or anti-blotin) |
| IgG* | protein A or protein G |
| 5-Bromo-2′-Deoxyuridine | specific antibodies |
| drug | drug receptor |
| toxin | toxin receptor |
| carbohydrate | lectin or carbohydrate receptor |
| peptide | peptide receptor |
| protein | protein receptor |
| enzyme substrate | enzyme |
| DNA (RNA) | aDNA (aRNA)** |
| hormone | hormone receptor |
| ion | chelator |
| lac repressor protein Lac I | lac operator (LacOP) |

*IgG is an immunoglobulin.
**aDNA and aRNA are the antisense (complementary) strands used for hybridisation.

A competition assay may also be employed wherein antibodies specific to EPSTI1 are attached to a solid support and labelled EPSTI1 and a sample derived from the host are passed over the solid support and the amount of label detected attached to the solid support can be correlated to a quantity of EPSTI1 in the sample.

The described EPSTI1 specific antibodies may also be used in an immunohistochemical assay to detect or quantify the presence of EPSTI1 in a tissue sample. The finding that the EPSTI1 gene is highly expressed in tissues characterised by extensive epithelial-stromal interaction, In particular in cancerous tissues, suggest that immunohistochemical analysis of EPSTI1 expression may reveal important molecular events associated with organ development, tissue remodelling and neoplasia and thus prove to be an important diagnostic and prognostic tool.

All the above described analyses may be employed on samples isolated from tissues selected from the group primarily consisting of breast tissue, placenta tissue, thymus, lung, stomach, thymus, prostate, adrenal gland, pancreas, lymphoid tissue, liver, uterus, small intestine, spleen, salivary gland, testes, colon, skin and extracellular body fluids, however other tissues may be considered as well, and the method of detecting the presence of detectable EPSTI1 polypeptide or mRNA in the test sample indicates that the test subject has or is at risk of developing metastatic cancer. Said metastatic cancer may primarily be selected from the group consisting of breast cancer, cancer of the male and female genital tract, and cancer of the thymus, lung, stomach, small intestine, prostate, adrenal gland, pancreas, colon, pancreas, lymphoid tissue, liver, salivary gland, spleen and skin.

Lymphoid tissue comprises lymph, lymph nodes, the spleen, the thymus, Peyer's patches, adenoids and pharyngeal tonsils. The predominant cell types of the lymphoid tissue is lymphocytes such as B-lymphocytes and T-lymphocytes.

As is the case with stromelysin-3, one of the few other genes which like EPSTI1 is directly involved in epithelial-stromal interaction, such genes may have Important implications for future strategies of diagnosis, prognosis and treatment of cancer. Stromelysin-3, which was originally reported to be overexpressed in the stroma of breast carcinomas has now been established as an independent prognostic marker of malignancy (Engel, G., et al. (1994) Int. J. Cancer 58: 830-835; Ahmad, A., et al. (1998) Am. J. Pathol. 152: 721-728.). As is the case of stromelysin-3 also EPSTI1 is highly expressed in normal placenta (example 1 and 5). Thus the elements of the present invention may be used in a prognostic in vitro assay or may be used in a diagnostic in vitro assay.

Therefore, the present invention is furthermore directed towards the diagnosis of malignant cancer by detection of the EPSTI1 mRNA or the EPSTI1 protein encoded by the EPSTI1 gene. The present invention thus contemplates the use of recombinant EPSTI1 DNA and antibodies directed against the EPSTI1 protein to diagnose the metastatic potential of several types of tumour cells, including, for example, breast, genital tract, thymus, lung, stomach, small intestine, prostate, adrenal gland, pancreas, colon, pancreas, lymphoid tissue, liver, salivary gland, spleen and skin and similar tumour cells.

The present Invention provides a new method for diagnosing metastatic cancer and for distinguishing metastatic or invading tumours from benign tumours. In particular, the present invention demonstrates a property of the herein described mammalian gene, EPSTI1, whose expression is about 10 to at least 160 fold higher in metastatic tumour cells than in the corresponding normal cells, such as 20 fold higher, e.g. 30 fold higher, e.g. 40, e.g. 45, e.g. 50, e.g. 55, e.g. 60, e.g. 65, such as 70, such as 75, e.g. 80 fold higher, e.g. 90, e.g. 95, such as 100 fold higher, e.g. 105, e.g. 110, e.g. 115, e.g. 120, such as 125 fold higher, e.g. 130, e.g. 135, e.g. 140, such as 145, e.g. 150, e.g. 155, such as 160 fold higher. According to the present invention metastatic cancer of can be detected in patient's body fluids e.g. blood, serum or plasma by a simple immunoassay. Moreover, metastatic cancer can also be diagnosed in tissue biopsies by the present immunoassays or by in situ hybridization assays.

Metastasis is the formation of secondary tumours by cells derived from a primary tumour. The metastatic process Involves mobilization and migration of primary tumour cells from the site of the primary tumour into new tissues where the primary tumour cells Induce the formation of secondary (metastatic) tumours. In accordance with the present inventive discovery, the increased expression of the EPSTIL gene in a cell or tissue is strongly indicative of metastatic potential. The present invention utilises this correlation of high mammalian EPSTI1 gene expression with high metastatic potential to detect or diagnose malignant cancer. Both the mammalian EPSTI1 nucleic acid and antibodies directed against mammalian EPSTI1 proteins are contemplated for use in the diagnosis of malignant cancer.

The present invention is also directed to the detection of metastatic cancer in tissue specimens by use of the EPSTI1 DNA as a nucleic acid probe for detection of EPSTI1 mRNA, or by use of antibodies directed against the EPSTI1 protein.

The nucleic acid probe of the present invention may be any portion or region of a mammalian EPSTI1 RNA or DNA sufficient to give a detectable signal when hybridized to EPSTI1 mRNA derived from a tissue sample. The nucleic acid probe produces a detectable signal because it is labelled in some way, for example because the probe was made by incorporation of nucleotides linked to a “reporter molecule”.

A “reporter molecule”, as used in the present specification and claims, is a molecule which, by its chemical nature, provides an analytically identifiable signal allowing detection of the hybridized probe. Detection may be either quantitative or quantitative. The most commonly used reporter molecules in this type of assay are either enzymes, fluorophores or radionuclides covalently linked to nucleotides which are incorporated into a EPSTI1 DNA or RNA. Commonly used enzymes include horseradish peroxidase, alkaline phosphatase, glucose oxidase and α-galactosidase, among others. The substrates to be used with the specific enzymes are generally chosen for the production, upon hydrolysis by the corresponding enzyme, of a detectable colour change. For example, p-nitrophenyl phosphate is suitable for use with alkaline phosphatase conjugates; for horseradish peroxidase, 1,2-phenylenediamine, 5-aminosalicyclic acid or tolidine are commonly used.

Incorporation into a EPSTI1 DNA probe may be by nick translation, random oligo priming, by 3′ or 5′ end labelling, by labelled single-stranded DNA probes using single-stranded bacteriophage vectors (e.g. M13 and related phage), or by other means, (Sambrook et al., 1989, Molecular Cloning, A Laboratory Manual. Cold Spring Harbor Laboratory Press. Pages 10.1-10.70). Incorporation of a reporter molecule into a EPSTI1 RNA probe may be by synthesis of EPSTI1 RNA using T3, T7, Sp6 or other RNA polymerases (Sambrook et al., supra: 10.27-10.37).

Detection or diagnosis of metastatic cancer by the nucleic acid probe of the present invention can be by a variety of hybridization techniques which are well known in the art. In one embodiment, patient tissue specimens are sectioned and placed onto a standard microscope slide, then fixed with an appropriate fixative. The EPSTI1 RNA or DNA probe, labelled by one of the techniques described above, Is added. The slide is then incubated at a suitable hybridization temperature (generally 37° C. to 55° C.) for 1-20 hours. Non-hybridized RNA or DNA probe is then removed by extensive, gentle washing. If a non-radioactive reporter molecule is employed in the probe, the suitable substrate is applied and the slide incubated at an appropriate temperature for a time appropriate to allow a detectable colour signal to appear as the slide is visualized under light microscopy. Alternatively, if the EPSTI1 probe is labelled radioactively, slides may be dipped in photoemulsion after hybridization and washing, and the signal detected under light microscopy after several days, as exposed silver grains.

Metastatic cancer can also be detected from RNA derived from tissue specimens by the EPSTI1 nucleic acid probe. RNA from specimens can be fixed onto nitrocellulose or nylon filters, and well-known filter hybridization techniques may be employed for detection of EPSTI1 gene expression. Specimen mRNA can be purified, or specimen cells may be simply lyzed and cellular mRNA fixed onto a filter. Specimen mRNA can be size fractionated through a gel before fixation onto a filter, or simply dot blotted onto a filter.

In another embodiment, the EPSTI1 nucleic acid detection system of the present invention also relates to a kit for the detection of EPSTI1 mRNA. In general, a kit for detection of EPSTI1 mRNA contains at least one EPSTI1 nucleic acid. Such an EPSTI1 nucleic acid can be a probe having an attached reporter molecule or the EPSTI1 nucleic acid can be unlabelled. The unlabelled EPSTI1 nucleic acid can be modified by the kit user to include a reporter molecule or can act as a substrate for producing a labelled EPSTI1 probe, for example by nick translation or RNA transcription.

In another embodiment, the kit is compartmentalized: a first container can contain EPSTI1 RNA at a known concentration to act as a standard or positive control, a second container can contain EPSTI1 DNA suitable for synthesis of a detectable nucleic acid probe, and a third and a fourth container can contain reagents and enzymes suitable for preparing said EPSTI1 detectable probe. If the detectable nucleic acid probe is made by incorporation of an enzyme reporter molecule, a fifth or sixth container can contain a substrate, or substrates, for the enzyme provided.

The EPSTI1 mRNA may be reverse transcribed into cDNA in any of the herein described detection methods based on the detection of an EPSTI1 transcript.

In accordance with the present invention, as described above, the EPSTI1 protein or portions thereof can be used to generate antibodies useful for the detection of the EPSTI1 protein in clinical specimens. Such antibodies may be monoclonal or polyclonal. Additionally, it is within the scope of this invention to include second antibodies (monoclonal or polyclonal) directed to the anti-EPSTI1 antibodies. The present invention further contemplates use of these antibodies in a detection assay (immunoassay) for the EPSTI1 gene product.

One embodiment of the present invention is directed to a method for diagnosing metastatic cancer by contacting or applying an antibody reactive with an EPSTI1 polypeptide to a tissue or blood sample taken from an individual to be tested for metastatic cancer. Formation of an antigen-antibody complex In this immunoassay is diagnostic of metastatic cancer.

In a preferred embodiment, the present invention provides a method for diagnosing metastatic cancer which involves contacting body fluids such as e.g. blood, serum or plasma from an individual to be tested for such cancer with an antibody reactive with a mammalian EPSTI1 protein or an antigenic fragment thereof, for a time and under conditions sufficient to form an antigen-antibody complex, and detecting the antigen-antibody complex.

The presence of the EPSTI1 protein, or its antigenic components, in a patient's serum, tissue or biopsy sample can be detected utilizing antibodies prepared as above, either monoclonal or polyclonal, in virtually any type of immunoassay. A wide range of immunoassay techniques are available as can be seen by reference to Harlow, et al. (Antibodies: A Laboratory Manual, Cold Spring Harbor Press, 1988) and U.S. Pat. Nos. 4,016,043 and 4,424,279. This, of course, includes both single-site and two-site, or “sandwich” of the non-competitive types, as well as in traditional competitive binding assays. Sandwich assays are among the most useful and commonly used assays. A number of variations of the sandwich assay technique exist, and all are intended to be encompassed by the present invention. Briefly, in a typical forward assay, an unlabelled antibody is immobilized in a solid substrate and the sample to be tested brought into contact with the bound molecule. After a suitable period of incubation, for a period of time sufficient to allow formation of an antibody-antigen binary complex, a second antibody, labelled with a reporter molecule capable of producing a detectable signal is then added and incubated, allowing tie sufficient for the formation of a ternary complex of antibody-labelled antibody. Any reacted material is washing way, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody, or a reverse assay in which the labelled antibody and sample to be tested are first combined, incubated and then added to the unlabeled surface bound antibody. These techniques are well known to those skilled In the art, and then possibly of minor variations will be readily apparent. As used herein, “sandwich assay” is intended to encompass all variations on the basic two-site technique.

The EPSTI1 protein may also be detected by a competitive binding assay in which a limiting amount of antibody specific for the EPSTI1 protein is combined with specified volumes of samples containing an unknown amounts of the EPSTI1 protein and a solution containing a detectably labelled known amount of the EPSTI1 protein. Labelled and unlabeled molecules then compete for the available binding sites on the antibody. Phase separation of the free and antibody-bound molecules allows measurement of the amount of label present in each phase, thus indicating the amount of antigen or hapten in the sample being tested. A number of variations in this general competitive binding assays currently exist.

In any of the known immunoassays, for practical purposes, one of the antibodies or the antigen will be typically bound to a solid phase and a second molecule, either the second antibody in a sandwich assay, or, in a competitive assay, the known amount of antigen, will bear a detectable label or reporter molecule in order to allow visual detection of an antibody-antigen reaction. When two antibodies are employed, as in the sandwich assay, it is only necessary that one of the antibodies be specific for the EPSTI1 protein or its antigenic components. The following description will relate to a discussion of a typical forward sandwich assay; however, the general techniques are to be understood as being applicable to any of the contemplated immunoassays.

In the typical forward sandwich assay, a first antibody having specificity for the EPSTI1 protein or its antigenic components is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing the molecule to the insoluble carrier. Following binding, the polymer-antibody complex is washed in preparation for the test sample. An aliquot of the sample to be tested is then added to the solid phase complex and incubated at a suitable temperature ranging from about 4° C. to about 37° C. (for example 25° C.) for a period of time sufficient to allow binding of any sub-unit present in the antibody. The incubation period will vary but will generally be in the range of about 2-40 minutes to several hours. Following the incubation period, the antibody sub-unit solid phase Is washed and dried and incubated with a second antibody specific for a portion of a EPSTI1 hapten. The second antibody is linked to a reporter molecule which is used to indicate the binding of the second antibody to the hapten.

By “reporter molecule”, as used in the present specification and claims, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphates, among others. The substrates to be used with the specific enzymes are generally chosen for the production, upon hydrolysis by the corresponding enzyme, of a detectable colour change. For example, p-nitrophenyl phosphate is suitable for use with alkaline phosphatase conjugates; for peroxidase conjugates, 1,2-phenylenediamine, 5-aminosalicyclic acid, or tolidine are commonly used. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the ternary complex of antibody-antigen-antibody. The substrate will react with the enzyme linked to the second antibody, giving a qualitative visual signal, which may be further quantitated, usually spectrophotometrically, to give an indication of the amount of hapten which was present in the sample.

Alternately, fluorescent compounds, such as fluorescein and rhodamine, may be chemically coupled to antibodies without altering their binding capacity. When activated by illumination with light of a particular wavelength, the fluorochrome-labelled antibody absorbs the light energy, inducing a state of excitability in the molecule, followed by emission of the light at a characteristic colour visually detectable with a light microscope. The fluorescent labelled antibody is allowed to bind to the first antibody-hapten complex. After washing off the unbound reagent, the remaining ternary complex is then exposed to the light of the appropriate wavelength, the fluorescence observed Indicates the presence of the hapten of interest. Immunofluorescence techniques are very well established in the art. However, other reporter molecules, such as radioisotope, chemiluminescent or bioluminescent molecules, may also be employed. It will be readily apparent to the skilled technician how to vary the procedure to suit the required purpose.

In another embodiment, the antibodies directed against the EPSTI1 protein may be incorporated into a kit for the detection of the EPSTI1 protein. Such a kit may encompass any of the detection systems contemplated and described herein, and may employ either polyclonal or monoclonal antibodies directed against the EPSTI1 protein. Both EPSTI1 antibodies complexed to a solid surface described above or soluble EPSTI1 antibodies are contemplated for use in a detection kit. A specific example of such a kit may be an ELISA kit.

A kit of the present invention has at least one container having an antibody reactive with a mammalian EPSTI1 polypeptide. However, the present kits can have other components. For example, the kit can be compartmentalized: the first container contains EPSTI1 protein as a solution, or bound to a solid surface, to act as a standard or positive control, the second container contains anti-EPSTI1 primary antibodies either free in solution or bound to a solid surface, a third container contains a solution of secondary antibodies covalently bound to a reporter molecule which are reactive against either the primary antibodies or against a portion of the EPSTI1 protein not reactive with the primary antibody. A fourth and fifth container contains a substrate, or reagent, appropriate for visualization of the reporter molecule.

Furthermore, a kit may contain directions for correlating whether binding, if any, or the level of binding, to said binding molecule is indicative of the individual mammal having a significantly higher likelihood of having metastatic cancer or a predisposition for having metastatic cancer.

The subject invention therefore encompasses polyclonal and monoclonal antibodies useful for the detection of EPSTI1 protein as a means of diagnosing metastatic cancer. Said antibodies may be prepared as described above, then purified, and the detection systems contemplated and described herein employed to implement the subject invention. The antibody or antigen binding fragment thereof may either be packaged in an aqueous medium or in lyophilized form.

The detection of a transcript with similarity to EPSTI1 in a lymphoma (B-cell non-Hodgkins lymphoma, NCBI accession no. AJ414515) may suggest that the gene is expressed In cells of lymphoid origin in general and thus, epstil may be involved in immunological functions. Therefore, substantially pure epstil polypeptide, modified epstil polypeptide or reagents interfering with epstil polypeptide may be used as treatment for multiple immunological disorders, including for instance psoriasis, arthritis and leukemia. Likewise, the identification of ESTs representing EPSTI1 in SAGE libraries (Unigene cluster id: Hs 343800) including microvascular endothelial cells, primary ovary carcinoma, colon adenocarcinoma, gastic carcinoma and neoplastic pancreas may implicate EPSTI1 gene expression in diseases and disorders of these tissues in general. Therefore, substantially pure epstil polypeptide, modified epstil polypeptide or reagents interfering with epstil polypeptide may be used as treatment for vascular diseases such as teleangiectasia, atherosclerosis, diseases of the uro-genital tract in general, including endometriosis; gastic ulcers and diabetes.

Finally, the fact that the stroma of an invasive carcinoma exhibited an elevated, albeit modest, EPSTI1 expression may implicate EPSTI1 In proliferative disorders of connective tissue. Therefore, substantially pure epstil polypeptide, modified epstil polypeptide or reagents interfering with epstil polypeptide may be used as treatment for diseases/disorders of connective tissue, including for Instance hypertrophic scar, scleroderma, keloids and systemic sclerosis.

Furthermore, the present application disclose a general method for isolation of nucleic acid sequences coded by genes which are regulated by the interaction between epithelial cells and the surrounding stroma cells, the method comprising:

a) extracting RNA from epthelial cells and stroma cells cultured as a co-culture in a three-dimensional culture system and from epithelial cells and stroma cells cultured as separate cultures in a similar three-dimensional culture system.

b) selecting two or more marker genes which are specific for the epithelial cell-lineage and the stroma cell-lineage, respectively.

c) determining the mRNA level of said cell-lineage specific markers in the RNA extracted from the co-culture as well as in the RNA extracted from the separate cultures of epithelial cells and stroma cells.

d) normalising the RNA extracted from the separate cultures by mixing (pooling) the RNA from the separate cultures to obtain ratios of the level of cell-lineage specific marker mRNAs that are similar to the ratios observed in the RNA isolated from the co-culture.

e) identifying transcripts or cDNA copies of transcripts which are differently representated In the RNA extracted from the co-culture relative to the normalised (pooled) RNA from separate cultures.

f) isolating said transcripts or cDNA copies of transcripts.

One preferred type of three-dimensional culture is described in example 1, however other types of three-dimensional cultures allowing the interaction between epithelial and stromal cells are comtemplated. One such example is the matrigel plug assay (Kawaguchi et al. Proc Natl Acad Sci USA 1998, 95:1062-1066). In example 1 cytokeratin 19, vimentin and thy-1 are used as cell-lineage marker genes, however other cell-liniage specific markers have been described in the literature and can be applied to complement or substitute cytokeratin 19, vimentin and thy-1. Non-limiting examples of additional cell-lineage specific markers are: sialomucin, E-cadherin, epithelial specific antigen (ESA), other cytokeratins, as markers of the epithelial cell-lineage and fibroblast activation protein (Mersmann et al., Int. J. Cancer. 2001, 92:240-248) as marker of the stromal cell-lineage.

The differentially expressed transcripts may be identified by a number of methods. One preferred method is the method of differential display (Liang and Pardee (1992) Science 257:967-71) and later developments thereof. However, a number of other methods e.g. various differential cloning technologies (Ausubel et al. (2000) or one of a variety of different array techniques (see, e.g., Lockhart et al., Nature Biotechnology (1996) 14: 1675-1680, Shena et al., Science (1995) 270: 467-470 and In WO 98/51789) can be applied. Finally the method implies that the identified transcripts are isolated. Depending on the particular method used for identification of the differentially expressed transcripts the skilled artisian would most likely select a procedure, which is based on recombinant gene technology. A treatise of this subject can be found in Ausubel et al. (2000) and Sambrook et al, (1989), both of which are incorporated by reference. One preferred procedure that is based upon the PCR technique is described in example 1.

Most human cancers emerge from epthelial cells, thus one particular interesting type of epithelial cells are cancer cells. However, the present invention is not limited to cancer cells. Similarly one preferred type of stromal cells are fibroblasts In particular (human telomerase (hTERT) transduced) normal breast fibroblasts, but the interaction between other cell types and stroma Is also contemplated. Thus, other models of interaction may include invasion of a fibrin gel or the like by placental trophoblasts (or cell lines) and spouting of endothellal cells (or cell lines, HUVEC) in a collagen gel or the the like. The former models placental development and the latter models angiogenesis, which are both normal, controlled processes, which display characteristics of invasive cancer.

### EXAMPLES

### Example 1

### Identification, Isolation and Preliminary Characterisation of the EPSTI1 Gene

To identify changes in gene expression during direct epithelial-stromal interaction in a 3D tumour environment assay, tumour cells and fibroblasts were cultured either separately or in combination with one another (Rønnov-Jessen, et al. (1995). J. Clin. Invest. 95: 859-873; Rønnov-lessen et al. (1992) In Vitro Cell. Dev. Biol. 28A: 273-283), and total RNA was extracted after ten days of incubation. Prior to differential display analysis the samples were normalized by mixing RNA from fibroblasts and from tumour cells cultured separately in the 3D tumour environment assay in a ratio which match the ratios of selected mRNA's in the co-culture as evaluated by real time PCR of lineage-specific markers. The normalization criteria were as follows: Identical expression levels of two housekeeping genes, GAPDH and TATA box binding protein (TBP), expressed in both cell types combined with Identical expression levels of two fibroblast markers, vimentin and thy-1, and one epithelial-specific marker, cytokeratin 19.

3D Tumour Environment Assay and RNA Isolation.

2 ml Collagen gels were prepared in 6 well dishes (Nunc, Roskilde, Denmark) as previously described (Rønnov-Jessen et al., 1992; Rønnov-Jessen et al., 1995) at a final concentration of 2.4 mg/ml. Prior to gelification, 2.5×10⁵ MCF-7 (Rønnov-Jessen et al., 1992) or 3.0×10⁵ D533 (hTERT-transduced normal breast fibroblasts, see below) (Nielsen et al., to be published elsewhere) were added to separate gels or combined in one gel. Culture medium (Dulbecco's modified Eagle medium 1885; GibcoBRL product #31885, Life Technologies, Taastrup, Denmark) supplemented with final concentrations of 7 non-essential amino acids, 2 mM L-glutamin (G3126, Sigma, Vallensbaek, Denmark), 5% fetal calf serum (mycoplasma screened australian serum, purchased from Life Technologies, Taastrup, Denmark), 6 ng/ml insulin (Boehringer Mannheim c/o Ercopharm, Kvistgaard, Denmark) and 50 mg/ml gentamycin (gentamycin sulphate, Biological Industries, Haemek, Israel) was added and changed three times a week. After 10 days of incubation in a Heraeus incubator in a humified atmosphere of 75% N₂, 20% O₂, 5% CO₂, total RNA was extracted with TRIZOL® Reagent (GibcoBRL, Life Technologies, Taastrup, Denmark) according to the manufacturer's instructions, and RNA extracted from the cells cultured in separate were pooled in a ratio normalized with respect to the levels of specific markers as described.

Cell Lines and Breast Biopsies.

Cell lines included MCF-10A, MCF-7 S9, HMT-3909, HMT-3522, T47D, ZR-75, BT-20, MDA-MB-435 (for original references, see (Rønnov-Jessen et al., 1996)) and sorted luminal and myoepithelial cells (Pechoux et al., 1999). D533 was obtained by transduction of primary breast fibroblasts with human telomerase (Rønnov-Jessen, et al. (1990) Lab. Invest. 63: 532-543; Morales, C. P., et al. (1999) Nature Genetics 21: 115-118.). Cells were Infected with retrovirus supernatant containing the catalytic sub-unit pBABE-hTERT (a gift from Dr. Judith Campesi, Life Sciences Division, Lawrence Berkeley National Laboratory, Berkeley, Calif., USA) in presence of 8 μg/ml polybrene (Sigma, Albertslund Denmark). Infected cells were selected in presence of 0.7 μg/ml pyromysin (Puromycin dihydrochloride, P7255, Sigma).

The procedure for collection of biopsies of normal breast (n=5) and Invasive breast carcinomas (n=8) was reviewed by the Regional Scientific-Ethical Committees for Copenhagen and Frederiksberg, Denmark and found consistent with danish Laws no. 503 and 24th Jun. 1992 and no. 499 of 12th Jun. 1996. Immediately after surgical removal, the tissue was frozen in n-Hexan on dry ice and stored at −80° C. until use. The tissue was crushed with a mortar cooled with liquid nitrogen and RNA was extracted as above. Normal breast organoids from normal breast biopsies were isolated as previously described (Rønnov-Jessen and Petersen, 1993), and subsequently used for RNA Isolation. Fibroblasts were explanted In serum-free medium, and myofibroblasts were generated by stimulation with 20% fetal calf serum as described (Rønnov-Jessen and Petersen, (1993). Lab. Invest. 68: 696-707.)

Differential Display and Sequencing.

Prior to differential display the RNA extracted from fibroblasts and tumour cells cultured separately was mixed in a ratio, which matched the contribution of selected mRNA's from the respective cell types in co-culture as determined by real-time PCR analysis of the expression of two housekeeping genes and three lineage-specific markers (see below). For RT-PCR and differential display (DD), total RNA samples were treated with DNase I (18068-015, GibcoBRL, Ufe Technologies, Taastrup, Denmark) to remove any possible DNA contamination. RT-PCR and DD-PCR were performed using the HIEROGLYPH™ mRNA profile kit (Genomyx Corporation, Foster City, Calif.) which includes 12 oligo(dT) anchored T7 3′ primers (5′ACGACTCACTATAGGGCTTTTTTTTTTTTXX3′, SEQ ID NO. 3) and 20 arbitrary M13r 5′ primers (5′ACAATTTCACACAGGA(10×) 3′, SEQ ID NO. 4) which in combination cover up to 95% of the entire mRNA pool. For RT-PCR, 2 μl of total RNA (0.1 μg/μl) measured spectrophotometrically at OD 260 was mixed with 2 μl anchored primer (2 μM), and incubated at 65° C. for 5 min in a thermal cycler with a heated lid (PTC™-100, MJ Research, Struers KEBO Lab, Albertslund, Denmark), and cooled on ice. 16 μl of a core mix containing a final concentration of 1×SuperScript II RT buffer (18064-14, GIbcoBRL, Life Technologies, Taastrup, Denmark), 25 μM dNTP mix (Boehringer Mannheim purchased from Ercopharm Roche, Hvidovre, Denmark), 10 mM DTT (GibcoBRL, Life Technologies, Taastrup, Denmark), 1Unit/μl RNasin (N2511, Promega, purchased from Bie & Berntsen, Rødovre, Denmark) and 2Units/μl SuperScript II RT enzyme (GibcoBRL, Life Technologies, Taastrup, Denmark) was added per tube, and RT was run in the thermal cycler at 25° C. for 10 min, 42° C. for 60 min, 70° C. for 15 min followed by hold at 4° C. In each experiment two control samples without RT enzyme were included. The following DD-PCR was carried out in duplicate. For each sample, 2 μl of the arbitrary primer (2 μM) was mixed with 2 μl RT mix, and a PCR core mix was prepared for each anchored primer containing a final concentration of 1×PCR buffer (15 mM MgCl₂), 20 μM dNTP mix, 0.2 μM anchored primer, 0.05 Unit/μl Taq DNA polymerase (Boehringer Mannheim c/o Ercopharm, Kvistgaard, Denmark) and 0.125 μCi/μl (α-³³P)dATP (AH9904, Amersham Pharmacia Biotech, Hørsholm, Denmark). 16 μl PCR core mix was added per tube, and DD-PCR was performed at 95° C. for 2 min, a first segment of 4 cycles at 92° C. for 15 sec, 46° C. for 30 sec, 72° C. for 2 min, were followed by 25 cycles at 92° C. for 15 sec, 60° C. for 30 sec, 72° C. for 2 min, followed by 7 min at 72° C. and hold at 4° C. Samples were mixed with Stop Solution (US70724, USB Corporation, purchased from Amersham Pharmacia Biotech, Hørsholm, Denmark), heat denatured and loaded on a 6% denaturing polyacrylamide gel (HR-1000™, Genomyx Corporation, Foster City, Calif., USA), and run in a GenomyxLR™ programmable DNA sequencer apparatus (Genomyx Corporation, Foster City, Calif., USA) at 40° C., 800 V, 100 W for 16 hours. Following electrophoresis, the gels were washed and dried and exposed over night (Kodak BioMax MR film, Kodak, Glostrup, Denmark).

Differentially expressed bands were cut out with a scalpel, bidirectionally reamplified with a full-length T7 promoter 22-mer primer (5′GTAATACGACTCACTATAGGGC3′, SEQ ID NO. 5, DNAtechnology, Aarhus, Denmark) and a full-length M13 reverse (−48) 24-mer primer (5′AGCGGATAACAATTTCACACAGGA3′, SEQ ID NO. 6, DNAtechnology, Aarhus, Denmark) using Expand™ High Fidelity PCR System (Boehringer Mannheim c/o Ercopharm, Kvistgaard, Denmark) and purified with QIAquick Gel Extraction Kit (Struers KEBO Lab, Albertslund, Denmark) prior to automatic sequencing in an ABI PRISM 310 Genetic Analyzer (Perkin Elmer Applied Biosystems, Naerum, Denmark) using the ABI PRISM BigDye Terminator Cycle Sequencing Ready Reaction Kit (Elmer Applied Biosystems, Naerum, Denmark). The cDNA fragments were sequenced with M13 (−48) 24-mer reverse sequencing primer resulting in sequence information corresponding to the 3′ end of the mRNA.

Nucleotide sequences were used to search the National Center for Biotechnology Information database with the use of the BLASTN program (http://www.ncbi.nim.nih.gov/BLAST/; Altschul et al. (1990) J. Mol. Biol. 215:403-410; Madden et al. (1996) Meth. Enzymol. 266:131-141; Zhang, J. & Madden, T. L. (1997) Genome Res. 7:649-656). Differential expression was verified by real time PCR using gene-specific primers (see Table 1).

TABLE 1

Primers used for verification of differential
gene expression by real-time RT-PCR.

| | | |
|---|---|---|
| TARGET | primers 5′-3′ | SEQ ID NO. |

| | | |
|---|---|---|
| GAPDH | GAAGGTGAAGGTCGGAGT | 7 |
| | GAAGATGGTGATGGGATTTC | 8 |
| Tata box | GGCACCACTCCACTGTATCC | 9 |
| binding protein | GCACACCATTTTCCCAGAAC | 10 |
| Vimentin | CGAAAACACCCTGCAATCTT | 11 |
| | TTGGCAGCCACACTTTCATA | 12 |
| Thy-1 | AGCATCGCTCTCCTGCTAAC | 13 |
| | GCACGTGCTTCTTTGTCTCA | 14 |
| Cytokeratin 19 | GAGGTGGATTCCGCTCCGGGCA | 15 |
| | ATCTTCCTGTCCCTCGAGCAG | 16 |
| EPSTI1 | CTCTACTGCCAGGAAATGC | 17 |
| | GCCTGTAGCAGGATAGCTC | 18 |
| Adrenal gland | CTTTTTGCAGAGGCCAATA | 19 |
| protein | GTGCGACCGACTGGAATAAC | 20 |

5′ Rapid amplificaton of cDNA ends (5′RACE)

To obtain a full-length cDNA a 5′RACE experiment was performed.

5′RACE was performed using the system from Life Technologies (18374-041, Life Technologies, Taastrup, Denmark) according to the manufacturer's instructions. cDNA was synthesised using a gene specific primer GSP-1 5′GTAGGGATTAAAATCTAAAA 3′ (SEQ ID NO. 21). 1 μg DNA'se treated total RNA from normal breast organoids (see above) or human placenta (Clontech human total RNA master panel K4005-1, purchased from Becton Dickenson, Brøndby, Denmark) were used as template. cDNA purification and tailing of 5′ ends were performed according to the manufacturer's instructions. Tailed cDNA was amplified using a nested PCR strategy essentially performed as described by the manufactor. 20 pmol of each primer were used and the gene specific primers in the first and second round of PCR amplification were GSP-2 5′GGTCMGTGTGTGGGCAGTTG3′ (SEQ ID NO. 22) and GSP-3 5′CCAACAGCCTCCAGATTGCT3′ (SEQ ID NO. 23). The PCR reactions were performed in a 50 μl volume at a final concentration of 1×PCR buffer including 1.5 mM MgCl₂, 0.5 u hotstar taq polymerase (Quiagen, Merck Albertslund, Denmark) and 200 μM dNTP mix. The PCR conditions were 95° C. for 15 min, 35 cycles at 94° C. for 1 min, 57° C. for 1 min, 72° C. for 2 min, followed by 10 min at 72° C. and hold at 4° C. 2 μl PCR product from the first PCR round were used as a template in the second round of amplification. 20 μl PCR product was electrophoresed on a 1.5% agarose gel, the product was cut out, purified and sequenced as described above. The GSP-3 primer was used for sequencing.

To be sure to obtain full length cDNA, the 5′RACE experiment was repeated with a strategy favouring amplification of full length sequences using the Invitrogen GeneRacer Kit (L1502-1, Invitrogen, Taastrup, Denmark) based on an oligo capping method (Maruyama and Sugano, 1994). 5 μg of total RNA from normal breast organoids and 2 μg of total RNA from human placenta RNA were used. Dephosphorylation, decapping, RNA oligo ligation, phenol-chloroform extractions, ethanol precipitations and cDNA synthesis were all performed according to the manufacturer's instructions. The GSP-1 primer was used for cDNA synthesis, and 2 μl cDNA was used for nested PCR. New gene specific primers located 5′ for GSP-2 and GSP-3 were used. 1 μl GeneRacer™ 5′ Primer and 1 μl GeneRacer™ 5′ Nested Primer were used in the first and second PCR amplification round, respectively. In the first amplification round 10 pmol GSP-3 and GSP-4 5′CCCAGCTGTTACCGCTATTCA3′ (SEQ ID NO. 24) were used. In the second round of amplification 10 pmol of either GSP-5 5′GCTGCCGTTTCAGTTCCAGT3′ (SEQ ID NO. 25), GSP-6 5′GGTGAACCGGTTTAGCTCTG3′ (SEQ ID NO. 26), GSP-7 5′CTTCCACTrCTCCAGGTTGG3′ (SEQ ID NO. 27) or GSP-8 5′TTAGGGGCTGCCTCCAAAC3′ (SEQ ID NO. 28) were used. Otherwise the PCR conditions and product purification were identical to those described above. Sequencing was performed with the gene specific primer used in the last amplification round.

Characterization of Gene Expression

Throughout this study the level of specific mRNAs was quantified by real time PCR.

cDNA Synthesis and Real Time PCR

2 μg of total RNA isolated as described above or total RNA from 24 human tissues (Clontech human total RNA master panel K4005-1, purchased from Becton Dickenson, Denmark) were DNA'se treated (DNase I Amp Grade, Life Technologies, Taastrup, Denmark) and used as template for first strand synthesis with oligo dT primers in a 30 μl volume (SuperScript Preamplification System, Life Technologies, Taastrup, Denmark) according to the manufacturer's instructions.

The gene expression level was determined according to the real time PCR standard curve method (for review see (Bustin, (2000)). 1 μl of oligo dT based cDNA was used as a template in a 50 μl volume with a final concentration of 1×PCR buffer and 0.5 u taq polymerase (Roche, Hvidovre, Denmark), a 1:70,000 dillution of 10.000×SYBR green 1 (Molecular probes, purchased from Bie & Berntsen, Rødovre, Denmark), 7.5 mM MgCl₂, 200 μM dNTP and 200 nM of forward and reverse primers. The used primers are listed in Table 1. All primers were purchased as standard desalted miniprimers from TAG Copenhagen, Denmark. Primers were designed using the primer3 software (http://www-genome.wi.mit.edu/cgl-bin/primer/primer3_www.cgi; Steve Rozen, Helen J. Skaletsky (1998) Primer3. Code available at http://www-genome.wi.mit.edu/genome_software/other/primer3.html.).

Standard curves for Thy-1 and vimentin were generated by serial dilutions of 2 μl D533 cDNA. For TATA box binding protein (TBP), cytokeratin 19 and adrenal gland protein (AGP) serial dilutions of 2 μl MCF-7 cDNA were used as template. For EPSTI1 and GAPDH standard curve generation a serial dilutions of MCF-7 cDNA or plasmid preparations were used as a template. One plasmid preparation contained the IMAGE clone 1147947 IMAGp998M042879Q2 (RZPD, Berlin Germany, GeneBank accession number AA633203) which is identical to the 3′ end of EPSTI1. The other plasmid preparation Is a TOPO cloned GAPDH PCR product (TOPO cloning System, Invitrogen). The use of a plasmid preparation as template did not affect the PCR efficiency. The plasmid preparations were prepared as follows: An E. coli colony was grown overnight in a shaking incubator at 37° C. in 200 ml LB medium (1 litre contains log tryptone, 5 g yeast extract, 5 g NaCl, 1 ml 1M NaOH) supplemented with 50 μg/ml ampicillin (Sigma, Albertslund, Denmark) and plasmids were isolated using the Qiagen plasmid maxi kit according to the manufacturer's Instructions (Qiagen 12162, purchased from Merck Albertslund, Denmark).

Real time PCR reactions were performed on an ICycler (BioRad, Heriev Denmark), and the PCR conditions were 94° C. for 3 min, 40-45 cycles at 94° C. for 30 sec, 56° C. for 30 sec, 72° C. for 30 sec, followed by hold on 4° C. Fluorescence was detected at 72° C. Amplification plots, threshold cycles (Ct) and standard curves were generated using the ICycler software version 2.1.880 or version 2.3 (BioRad). Melting profiles were generated by increasing the temperature 0.5° C. every 30 seconds. The fluorescence were detected at each temperature. The −dF/dT were calculated in Excel (Microsoft Office 97) and plotted against the temperature; a single peak indicating the presence of PCR product without primer dimers. In all real time PCR reactions no primer dimer artefacts occurred, and only the true product was amplified as determined by a single peak on the melting curve, right product size by gel electrophoresis, and sequencing of the PCR product. All standard curves had a correlation coefficient between 0.96-0.99. No product occurred in negative controls. In all tested samples Ct values were below 32, thus the impact of unspecific signals was considered to be insignificant. The relative EPSTI1 expression level was calculated using GAPDH as an internal standard. The EPSTIL expression level in normal breast tissue were designated the value (reference sample) 1. The reference cDNA were used in all real time PCR runs. The relative expression in tested samples were calculated by dividing the normalised EPSTIL expression (the EPSTIL expression level divided with the GAPDH expression level) in the test sample with the normalised EPSTI1 expression in the reference sample.

Results

To identify changes in gene expression during direct epithelial-stromal Interaction In a tumour environment assay, tumour cells and fibroblasts were cultured in separate or in combination (FIG. 1A), and total RNA was extracted after ten days of incubation. RT-PCR, DD-PCR (differential display PCR) and fragment re-amplification were performed using the HIEROGLYPH mRNA profile kit. After electrophoresis, differentially expressed transcripts (FIG. 1B, box) were isolated, reamplified, purified and automatically sequenced. For verification RNAs from fibroblasts and tumour cells in separate were mixed in a ratio that matched the ratios of RNAs in the recombinant culture as evaluated by real time PCR of lineage specific markers. The normalisation criterion was identical expression levels of two housekeeping genes, GAPDH and TATA box binding protein, expressed in both cell types combined with identical expression levels of two fibroblast markers, vimentin and thy-1, and one epithelial-specific marker, cytokeratin 19 (FIG. 1C). Primers corresponding to adrenal gland protein mRNA, which was not differentially expressed, i.e. displayed bands of apparent equal intensity, was included as a control (FIG. 1D). The amplicon of differential abundance was tentatively taken to represent a novel gene, which we designated BRESI, later changed to epithelial stromal interaction 1 (breast), EPSTI1.

The successfully sequenced nucleotide sequences were used to search the NCBI database. A diffentially expressed amplicon of 580 bp amplified by primer combination AP9/ARP1 was identified as a double band by DD-PCR and represented an unknown gene matching genomic sequences previously unassigned to any known function. Using gene-specific primers differential expression of EPSTI1 was verified by real time PCR using SYBR green 1 for detection on normalised RNA samples (FIG. 1C).

The EPSTI1 is upregulated during direct epithelial-stromal interaction, as shown by the expression level in normal breast versus invasive breast carcinomas. EPSTI1 is upregulated up to 65 times (range 2.5-65) In all breast carcinomas tested (n=8) as compared to normal breast (n=5).

In a tissue mRNA panel the most prominent expression of EPSTI1 found in placenta (42 times the expression in normal breast tissue). Thus, it is evident that the herein described novel human gene Is expressed in tissues characterised by extensive epithelial-stromal interaction, and expression of this gene is a crucial event in invasion and metastasis of cancer.

A full length cDNA of 1508 bp was generated by 5′RACE (FIG. 2A). The most 5′ end of the sequence was identified with the 5′RACE oligo capping method (Maruyama and Sugano, 1994). This strategy favours amplification of full length transcripts by elimination of truncated mRNA. Identical sequences were obtained using RNA from normal breast tissue and placenta tissue. This sequence matched two genomic clones with genbank accession number AL445217 and AL137878 and several ESTs, but the entire EPSTIL gene represented by its full-length sequence has not been described.

In WO 99/38881 a nucleotide sequence is given (SEQ ID 74) which shows similarities to the EPSTI1 nucleotide sequence given herein. However, multiple differences up to base no. 284 of Seq id 74 and base no. 252 of EPSTI1 are found. The start codon ATG of EPSTI1 is in position 66-68, and thus, the deduced protein sequence of SEQ ID 74 WO 99/38881 is entirely different from epstil up to aa 87. In support of correct translation of the EPSTI1 nucleotide sequence described herein is the fact that the predicted amino acid sequence of EPSTI1 exhibits a high level of similarity with the mouse homologue, BAB30623, also within this region. Beyond base no. 284 of SEQ ID 74 of WO 99/38881 and base no. 252 of EPSTI1, the nucleotide sequences are almost identical. However, SEQ ID 74 of WO 99/38881 lacks a G in position 251 (corresponding to position 320 of EPSTI1) leading to a frameshift, the reported sequences thus exhibit crucial differences and do not translate into identical proteins.

GenBank acc. no. BG822216 discloses a nucleotide sequence which also shows an overall high similarity with the nucleotide sequence of EPSTI1. However, BG822216 lacks a T in position 197 (corresponding to position 196 in EPSTIL) leading to a frameshift (ttg, leucine at aa position 44 of EPSTI1 to tgg, tryptophan In BG822216). Beyond position 694 of BG822216 (corresponding to position 698 of EPSTI1) still well within the coding region, there are several differences. The BG822216 and EPSTI1 nucleotide sequences do not translate into identical amino acid sequences.

The cDNA has an 921 bp open reading frame encoding a protein of 307 amino acids. When searched against all available databases for sequence homology the deduced protein sequence was found to exhibit 64% overall homology to a putative mouse protein (NBCI accession no. BAB30623), which has not been characterised further (FIG. 2B).

EPSTI1 was mapped electronically to 13q14.2 by comparing the cDNA sequence to both two separate neighbour clones containing genomic DNA (AL445217 and AL137878) and to the human genome sequence. The genome BLAST search also revealed the exon-intron structure of EPSTI1 (FIG. 3 and Table 2). The EPSTI1 gene contains 11 exons spanning 104.2 kb on genomic DNA, with a start codon located in exon 1 and a stop codon in exon 11. All the sequences at the intron-exon junctions conformed to the consensus sequence for splicing boundaries (ag-gt rule).

To correlate the observed EPSTI1 expression In culture with the in vivo situation, the expression of EPSTI1 in invasive breast carcinomas was analysed by RT-PCR. Interestingly, all carcinomas tested expressed EPSTI1, but even more Importantly, when compared to normal breast by real time PCR, EPSTI1 was upregulated up to 65 times in carcinomas (range 2.5-65; FIG. 4A).

Furthermore, the expression of EPSTI1 in other organs revealed a relatively high expression in thymus, stomach, lung, small Intestine, spleen and a most prominent expression in placenta (42 times the expression level in normal breast) (FIG. 4B). In contrast to tissue samples, a panel of tumour cell lines and fibroblasts in monolayer culture exhibited levels of EPSTI1 which were all lower than the expression detected in normal breast organoids. Collectively, these data indicate that EPSTI1 is specifically expressed in epithelial-stromal interaction and that 3-dimensional interaction amplifies the expression.

### Example 2

### Chromosomal Localisation: Current NCBI Annotation has Revised the Chromosomal Localization of EPSTI1

Due to the recent annotation of the National Center for Biotechnology Information (NCBI) database EPSTI1 is now mapped in silico to chromosome 13q13.3 using NCBI map viewer (NCBI News, National Center for Biotechnology Information, Spring 2001) by comparing the cDNA sequence to both two separate neighbour clones containing genomic DNA (AL445217 and AL137878) and to the human genome sequence (FIG. 5). The genome BLAST search also confirmed the exon-intron structure of EPSTI1 (FIG. 6 and Table 2). The EPSTI1 gene contains 11 exons spanning 104.2 kb on genomic DNA, with a start codon located in exon 1 and a stop codon in exon 11. All the sequences at the intron-exon junctions conformed to the consensus sequence for splicing boundaries (ag-gt rule).

TABLE 2

Exon-intron boundary sequences of the human EPSTI1. Capital
letters represent exon sequences. *intron 5 was located in the
overlapping region of two neighbour genomic clones AL445217 and
AL137878 and the right intron size was not identified, thus,
the 10 kb includes intron 5, exon 6 and intron 6.

| | | | | | | |
|---|---|---|---|---|---|---|
| | | | | | | Intron |
| Exon | Size | CDNAn | | | Intron | length |
| # | (bp) | position | Splice acceptor | Splice donor | # | kb |

| | | | | | | |
|---|---|---|---|---|---|---|
| 1 | 253 | 1-253 | | CCAGAGGCGgtgagtatt | 1 | 21.3 |
| 2 | 59 | 254-312 | tccttttagCACAAGTGC | TACAAAGAAgtatgtata | 2 | 1.4 |
| 3 | 84 | 313-396 | ctggcttagTTGCGGAGC | GACGGGTAGgtaagcaag | 3 | 5.0 |
| 4 | 74 | 397-470 | tttcctaagGTGGAAGCC | AAGGAAAAGgtcagaatt | 4 | 0.7 |
| 5 | 84 | 471-554 | acaatttagCTAAAAAGA | AGAGAGAAGgtaaggagg | 5 | 10.0* |
| 6 | 74 | 555-628 | caatttcagAGCAATAAA | TCAGCAATAgtaagttaa | 6 | |
| 7 | 94 | 629-722 | ttctggtagCAAAACCGC | TCAACATGGgtaagtgaa | 7 | 27.5 |
| 8 | 84 | 723-806 | cctttacagGCCAGAAGC | CATCAAAAGgtacttaca | 8 | 8.8 |
| 9 | 74 | 807-880 | tgttcctagAGTGAATTA | ACACAGGAGgtaaaactg | 9 | 17.2 |
| 10 | 100 | 881-980 | gttttaaagGGTAAATAA | AACAGCTGGgtgagtttt | 10 | 6.5 |
| 11 | 528 | 981-1508 | ttcttccagGGTATATGA | | | |

Localization to 13 q was confirmed by PCR on human monochromosomal hybrids (Drwinga et al. (1993) Genomics 16: 311-314.) covering fragments of chromosome 13 (FIG. 5). Briefly, DNA samples of human monochromosomal somatic cell hybrids containing chromosome 12, 13 and fragments of chromosome 13 (DNA samples NA10868, NA11689, NA11766, NA11767, NA14050, NA11575 from NIGMS, Camden, N.J., USA) and cDNA from breast were used as templates for PCR with human specific EPSTI1 primers 5′CAGGAGTGACTGGCTTCTCC3′ (SEQ ID NO. 29) and 5′AAGACCCCCAAAGCTTTCAA3′ (SEQ ID NO. 30).

### Example 3

### Confirmation of Predicted Transcript Size

To characterise the EPSTI1 transcript further a northern blot were performed on RNA isolated from human skeletal muscle, colon, thymus, spleen, kidney, liver, small intestine and placenta. Furthermore the possibility of alternative splicing was investigated.

Methods

Northern blot: Briefly, a probe consisting of 0.8 kb from the coding region of EPSTI1 was labelled with (α-³²P)dATP by linear PCR (Strip-EZ PCR kit, Ambion, purchased from Intermedica, Sweden). A commercial multiple tissue Northern blot (BD Biosciences Clontech, Palo Alto, Calif.) was probed under high stringency, and washed according to the manufacturer's instructions. The blot was exposed to x-ray film (BioMax MS & TranScreen, Kodak) overnight.

Alternative splicing: was investigated performing a RT-PCR across the entire open reading frame. For RT-PCR across the entire ORF the EPSTI1 primers 5′ATGAACACCCGCAATAGAGTG3′ (SEQ ID NO. 31) and 5′AAGACCCCCAAAGCTTTCAA3′ (SEQ ID NO. 30) were used with carcinoma or placenta cDNA as a template.

Result

The predicted transcript size of 1.5 kb was been confirmed by the Northern blot (FIG. 7A). The possibility of alternative splicing was excluded by the RT-PCR across the entire open reading frame (FIG. 7B).

### Example 4

### Further Analysis of the Predicted Amino Acid Sequence and Motif Similarity

Screening of a non-redundant protein database revealed no match with any existing human amino acid sequence. Interestingly, however, the sequence was found to exhibit 64% identity and 77% similarity to a putative mouse protein (accession no. BAB30623). This sequence was deduced from a full-length cDNA (accession no. AK017174.1) identified as a part of the RIKEN cDNA sequencing project, and has not been characterized further. The sequence of the mouse homolog covers the first 219 amino acids of EPSTI1 (FIG. 8A). The homology between the two putative proteins is distributed throughout the entire sequence, but is more prominent in the sequence spanning amino acids 66 to 219 of EPSTI1.

The predicted EPSTI1 protein has a molecular mass of 35.4 kDa with no transmembrane domains. In Western blot analysis the molecular mass was determined to be approximately 41-43 kDa.

To further estimate the size of the epstil protein, an additional Western blot was performed.

Modified Western Blotting

MCF7 FLAG-EPSTI1 (as described in Example 7) cells with or without the tetracycline derivative, doxycycline (Sigma-Aldrich, Vallenbaek, Denmark) were added and cultured as described in Example 7 were washed in PBS and lysed in a buffer containing 20 mM Hepes buffer pH 8.0; 1% NP-40 (BDH Laboratory Supplies, purchased from Bie & Berntsen, Rødovre, Denmark); 10% glycerol (Merck, Albertslund, Denmark); 2.5 mM EDTA (Sigma-Aldrich, Vallensbaek, Denmark); 5.7 mM PMSF (Sigma-Aldrich, Vallensbaek, Denmark); 5 μg/ml aprotinin (Sigma-Aldrich, Vallensbaek, Denmark). After 10 min incubation on ice the lysate was centrifuged 13,000×g for 10 min. The total concentration of protein was determined using Bio-Rad DC Protein Assay (Bio-Rad, Herlev, Denmark) following the manufacturer's instructions and read on an EL800 Universal Microplate Reader (Bio-Tek Instruments, Inc. purchased from Boule Nordic AB, Kastrup, Denmark). 20 μg total protein of each sample was separated on a 4-20% Tris-Glycine PAGEr® Gold precast gel (BioWhittaker Molecular Applications purchased from Medinova Scientific A/S, Hellerup, Denmark) and transferred to an Immobilon™-P Transfer Membrane (Millipore, Bedford, Mass.) using Mini Trans-Blot® Electrophoretic Transfer Cell (Bio-Rad, Herlev, Denmark). The membrane was blocked in Tris-buffered saline (TBS) with 5% skimmed milk (Bio-Rad, Herlev, Denmark) and 0.05% Tween@20 (Merck, Albertslund, Denmark) and the antibodies were diluted in the same buffer. The primary antibody, ANTI-FLAG® M2 monoclonal antibody (Sigma-Aldrich, Vallensbaek, Denmark) was diluted 1:3000, the secondary antibody, rabbit anti-mouse (Z0259, Dako, Glostrup, Denmark) was diluted 1:50 and the tertiary antibody, monoclonal mouse PAP (P0850, Dako, Glostrup, Denmark) was diluted 1:100. Between antibody incubations the membrane was washed three times in TBS with 0.05% Tween®20. Immunosignals were detected using enhanced chemiluminescence reagent and exposed on Hyperfilm (Amersham Biosciences, Hørsholm, Denmark). Based on this procedure, the estimated size of the EPSTI1 protein can be narrowed down to between 40-42 kDa and is most likely approximately 41 kDa.

The discrepancy from the predicted molecular mass may result from post-translational events such as glycosylation. It contains no N-teminal signal sequence and is therefore predicted to be a non-secreted protein. The EPSTI1 architecture was determined using the SMART algorithm (Schultz et al. (2000) Nucleic Acids Res. 28: 231-234), and three coiled-coil regions were predicted in positions 74-101, 128-188 and 226-265, respectively (FIG. 6).

Structure of EPSTI1

Currently, protein structure prediction is usually done by homology modelling of known protein structures with sequence homology higher than 30% (Brenner et al. (1998) Proc. Natl. Acad. Sci. (USA) 95: 6073-6078). Search for domains within the EPSTI1 sequence by this criterion did not render any conserved residues. Also, search within the SWISS-MODEL 3-dimensional database could not identify any 3D homologues. To search for possible repeat sequences, the extra 88 C-terminal amino acids of EPSTI1 was aligned with the 219 overlapping amino acids of EPSTI1 and BAB30623. Within the second and third coiled-coil, a possible repeat region of 33 amino acids (from 230 to 262), which exhibited 61% identity and 73% similarity to EPSTI1 (from 146-178) and BAB30623 (from 143-175), respectively, was identified (FIG. 8B).

Bioinformatic tools used: The nucleotide sequence was analysed with the BLASTN algoritm, genome BLAST and map viewer at the National Center for Biotechnology Information website (http://www.ncbi.nim.nih.gov/BLAST/). The TIGR Human Gene Index (Quackenbush et al (2000) Nucleic Acids Res. 28: 141-145) was searched to identify EST clusters aligning with the differentially expressed 580-bp transcript. Gene2EST (http://woody.embl-heidelberg.de/gene2est/) was used to identify EPSTI1-aligning ESTS. PSI BLAST (Altschul, S. F., et al. (1997) Nucleic Acids Res. 25: 3389-3402) was performed for protein alignment. Transmembrane domains and signaling peptide sequences and secondary architecture of the putative translation product were predicted using the Simple Modular Architecture Research Tool (SMART) (Schultz et al. (2000) Nucleic Acids Res. 28: 231-234; http://smart.embl-heidelberg.de). The RPS-BLASTP (http://www.NCBI.nlm.nih.gov/BLAST), ProDom, PRINTS, and Pfam databases (http://motif.genome.ad.jp) were searched for conserved domains. Three-dimensional homologs were searched in the SWISS-MODEL database (http://www.expasy.ch/swissmod/SWISS-MODEL.html). Multiple sequence alignment was performed with the Ciustal W multiple alignment program and ajusted manually (http://www.ch.embnet.org/software/ClustalW.html), and shaded with the Boxshade 3.21 program (http://www.ch.embnet.org/software/BOX_form.html). In the present context the default values of the programs were used.

### EXAMPLE 5

### Further Assessment of EPSTI1 Expression Level

The relative expression of EPSTI1 in breast cancer as compared to normal breast has been further substantiated relative to the result presented in example 1 by triplicate analysis, correlation to two different internal controls (GAPDH and TATA box binding protein) and inclusion of more tumour samples (total 14 carcinomas).

Also the relative expression of EPSTI1 in a number of non-cancerous human tissues has been further substantiated by triplicate analysis and correlation to two different internal controls.

Materials and Methods

The samples were obtained and the EPSTI1 mRNA level was estimated by real time PCR as described in Example 1.

With regard to the EPSTI1 mRNA level In the non-cancerous human tissues the level was estimated by real time PCR as described previously in RNA extracted from 1: normal breast (reference sample), 2: lung, 3: trachea, 4: bone marrow, 5: small intestine, 6: spleen, 7: stomach, 8: thymus, 9 normal breast 10: prostate, 12: skeletal muscle, 13: adrenal gland, 14: pancreas, 15: salivary gland, 16: foetal brain, 17: foetal liver. 18: spinal cord 19: placenta, 20: brain, 21: heart, 22: kidney, 23: liver, 24: colon, 25: uterus and 26: testis.

Results

All carcinomas tested (14/14) expressed EPSTI1, but even more importantly, when compared to normal breast, EPSTI1 was upregulated up to 72 times in carcinomas (range 5.6-72.1, FIG. 9A). That EPSTI1 was indeed upregulated in tumour tissue was confirmed by analysis of another 6 tumour samples comprizing four primary breast carcinomas and two metastases (range 6.5-158.4). To ensure that the observed EPSTI1 expression levels were not due to a variation in GAPDH expression, TATA box binding protein, which has been used successfully by others as an internal control for breast carcinomas (Bleche et al. (1999) Clin. Chem. 45: 1148-1156), was included as an internal control in 8 of the carcinomas, and the range of relative EPSTI1 expression was confirmed.

Whereas muscle tissues, i.e. skeletal- and cardiac muscle, were virtually devoid of EPSTI1 expression, all other tissues expressed EPSTI1, and we found a relatively high expression in small intestine, spleen, salivary gland, testes and a most prominent expression in placenta (14.0 times the expression level in normal breast) (FIG. 9B).

The level of upregulation in placenta as described in Example 1 (42.0 times the expression level in normal breast, example 1, FIG. 4B) was based on a single-sample analysis as compared to the triplication analysis described in the present Example 5.

Thus, when referring to the results presented here and in the previous examples, it is clear that the herein described novel human gene is expressed in tissues characterised by extensive epithelial-stromal interaction, and expression of this gene probably is a crucial event in invasion and metastasis of cancer.

### Example 6

### EPSTI1 is Expressed Primarily in the Epithelial Compartment

To further resolve whether EPSTIL was expressed in the epithelial or the stromal compartment, or both, we Isolated tumour cells and myofibroblasts from primary tissue and compared the expression to normal breast epithelial organolds. Furthermore, the localization of EPSTI1 expression was addressed by laser-assisted microdissection of tumour- and stromal-tissue, respectively, from a primary breast carcinoma.

Materials and Methods

The tumour cells and myofibroblasts were isolated from primary tissue by collagenase treatment. Briefly, normal breast organoids, fibroblasts and tumour cells were isolated as previously described (Rønnov-Jessen and Petersen (1993) Lab. Invest. 68: 696-707; Petersen et al. (1992) Proc. Natl. Acad. Sci. USA 89: 9064-9068), and subsequently used for RNA isolation.

Laser-assisted microdissection of tumour and stromal tissue vas performed by Laser Pressure Catapulting. Briefly, for laser pressure assisted microdissection, 10 μm cryosections were mounted on a glass slide covered with a thin polyethylene membrane (P.A.L.M, Bernried, Germany). The sections were ethanol fixed, stained with methylgreen or hematoxylin. Areas of carcinoma cells or stroma were circumcised with a UV-laser Robot Microbeam (P.A.L.M) and subsequently catapulted in to the cap of a microfuge tube.

Approximately 2000 cells were collected. RNA was isolated immediately using the Stratagene Micro RNA kit (Stratagene, purchased from AH Diagnostics, Aarhus, Denmark), DNAse treatment and cDNA synthesis was performed as described above in a volume scaled down to 20 μl.

The EPSTI1 mRNA level was estimated by real time PCR as described in Example 1.

Results

As shown in FIG. 10A, only tumour cells exhibited an elevated level of EPSTIL expression (30.0 times the expression level in normal breast). Moreover, the localization of EPSTI1 expression was addressed by laser-assisted microdissection of tumour—and stromal tissue, respectively, from a primary breast carcinoma (FIGS. 10B and C). Prior to microdissection the relative EPSTI1 expression was 6.5 times the expression level in normal breast. In the microdissected samples, tumour cells as well as stromal cells (including fibroblasts, myofibroblasts and microvasculature) expressed EPSTI1 (5.5 and 3.1 times normal breast, respectively, FIG. 10D). Finally, two samples representing a primary tumour and a lymph node metastasis from the same individual was included. The metastasis was virtually devoid of residual lymphatic tissue, and exhibited an EPSTI1 expression level comparable to the primary lesion (158.4 and 122.2, respectively).

In conclusion, both the collagenase isolation procedure and the laser-assisted microdissection showed that the EPSTI1 primarily is expressed in the epithelial compartment.

### Example 7

### The Subcellular Localization of EPSTI1

The subcellular localization of EPSTI1 was analyzed in a human breast cell line, MCF7, by conditional expression of FLAG-tagged EPSTI1 using the tetracycline-repressive gene regulation system.

Materials and Methods

The coding region of EPSTI1 was tagged with the FLAG epitope in the C-terminus by PCR amplification and cloned into the pRevTRE vector (Clontech, purchased from Becton Dickenson, Denmark).

Briefly, 0,5 μl cDNA from human breast was PCR amplified in a 50 μl volume with the Expand™ High Fidelity PCR System (Roche, Hvidovre, Denmark), 200 nM of forward 5′CGGTCGACGCCACCATGAACACCCGCMTAGA3′ (SEQ ID NO. 32) and reverse 5′CCATCGATGGTCACTTGTCATCGTCGTCCTTGTAGTCTATACCCCAGCTGTTACC3′ (SEQ ID NO. 33) primers. The PCR conditions were 94° C. for 5 min, 5 cycles at 94° C. for 45 sec, 55° C. for 30 sec, 72° C. for 1 min, 25 cycles at 94° C. for 45 sec, 65° C. for 30 sec, 72° C. for 1 min, followed by 7 min at 72° C. and hold at 4° C. The PCR product was electrophoresed in 1.5% low melt agarose gel, purified with QIAquick Gel Extraction Kit (Qiagen, Merck, Albertslund, Denmark) and eluted In 40 μl H2O. 15 μl of the purified PCR product was digested for 18 hours at 37° C. with Cla I and Sal I in 60 μl 1×restriction digest buffer H (Roche, Hvidovre, Denmark). 14 pi loading buffer was added, and the digest was gel purified and eluted in 50 μl H₂O. 100 ng pRevTRE was digested for 2 hours at 37° C. and 15 minutes at 65° C. with cla I and sal I in 14 μl 1× restriction digest buffer H (Roche, Hvidovre, Denmark). 1.8 μl 10× dephosphorylation buffer and 2 μl Shrimp alcaline phosphatase (SAP; Roche, Hvidovre, Denmark) was added to the pRevTRE restriction digest and incubated for 10 minutes at 37° C. and 15 minutes at 65° C. 1.5 μl of digested, gel purified PCR product and 6 μl SAP treated pRevTRE were ligated in a 20 μl volume with Rapid ligation kit (Roche, Hvidovre, Denmark) according to the manufacturer's instructions. 6 μl of the ligation product was used to transform one shot TOP10 cells (InVitrogen, Tåstrup Denmark) according to the manufacturer's instructions. Insert containing colonies were identified by colony PCR: An E. coli colony with insert was grown overnight in a shaking incubator at 37° C. in 200 ml LB medium supplemented with 50 μg/ml ampicillin (Sigma, Vallensbaek Denmark) and plasmids were isolated using the Qiagen plasmid maxi kit according to the manufacturer's instructions (Qlagen 12162, purchased from Merck Albertslund, Denmark). That the insert was correct was confirmed by sequencing. MCF7 Tet-OFF™ cells (Clontech, purchased from Becton Dickenson, Denmark) were cultured in DMEM 1885 (Gibco BRL, purchased from Invitrogen, Tåstrup, Denmark) containing 10% Tet System Approved Fetal Bovine Serum (Clontech, purchased from Becton Dickenson, Denmark) supplemented with 2 mM L-glutamine and 100 mg/ml G418 (Gibco BRL, purchased from Invitrogen, Tåstrup, Denmark).

FLAG tagged EPSTI1 was transduced into MCF7 Tet-OFF cells using the RetroMax retroviral transduction assay as described by the manufacturer (Imgenex, San Diego, Calif.). Infected cells containing the pRevTRE-FLAG-EPSTI1 vector were selected by adding 400 μg/ml hygromycin B (Gibco BRL, purchased from Invitrogen, Tastrup, Denmark).

The resulting MCF7 FLAG-EPSTI1 cell line was cultured using the culture medium described above. EPSTI1 expression was repressed by the addition of 100 ng/ml of the tetracycline derivative, doxycycline (Sigma-Aldrich, Vallensbaek)). Briefly, subconfluent cultures of MCF7 FLAG-EPSTI1 cells with or without exposure to 100 ng/ml doxycyline for 4 days were washed i PBS and fixed for 15 minutes In 3.7% formalin at room temperature and washed 3 times in PBS. Cells were permeabilized in 0.1% Triton X-100 in PBS for 10 minutes and washed 3 times In PBS. The cells were blocked for 5 minutes in 10% Normal goat serum (Biological Industries, purchased from In Vitro, Frederiksberg, Denmark) and incubated for 30 minutes with 1:1000 ANTI-FLA® M2 monoclonal antibody (Sigma-Aldrich, Vallensbaek, Denmark), and 30 minutes with 1:25 FITC conjugated secondary antibody (1070-02 Southern Biotechnology, Birmingham, Ala., USA) and counterstained with 1 μg/ml propidium iodide (Molecular probes, purchased from Bie & Berntsen, Rødovre, Denmark). Between incubations cells were washed 3 times in PBS. Immunofluorescence was visualized using a Zeiss LSM 510 laser scanning microscope (Carl Zeiss, Jena, Germany).

For Western Blotting, lysates and conditioned media were separated on NuPAGE™ 10% Bis-Tris Gel (Invitrogen, Groningen, Netherlands) and transferred to a polyvinylidene difluoride membrane (Amersham Pharmacia, Hørsholm, Denmark). The membrane was blocked in phosphate-buffered saline (PBS) with 5% skimmed milk (Bio-Rad, Herlev, Denmark) and 0.1% Tween®20 (Merck, Albertslund, Denmark) and the antibodies were diluted in the same buffer. The primary antibody, ANTI-FLAG® M2 monoclonal antibody (Sigma, Vallensbaek, Denmark) was diluted 1:2000 and the secondary antibody, HRP-goat-anti-mouse (Dako, Glostrup, Denmark) was diluted 1:2000. Between antibody incubations the membrane was washed four times in PBS with 0.1% Tween®20. Immunosignals were detected using enhanced chemiluminescence reagent and exposed on Hyperfilm (Amersham Biosciences, Hørsholm, Denmark). As a control, the membrane was stripped and incubated with anti-beta-actin diluted 1:5000 (A-5441 Sigma, Vallensbaek, Denmark) and proceeded as described above.

Results

Immunocytochemical analysis of FLAG-EPSTI1 revealed that EPSTI1 exhibited at least three expression patterns: 1) exclusive expression in the nucleus, 2) expression in both the nucleus and the cytoplasm, and 3) sole expression in the cytoplasm (FIG. 11A). It has been described by others that nuclear proteins may shuttle between the nucleus and cytoplasm. Likewise, some proteins as for instance steroid receptors are translocated to the nucleus upon ligand binding. That FLAG-EPSTI1 is indeed regulated by doxycycline was confirmed by Western blotting (FIG. 11B). Moreover, FLAG-EPSTI1 was not detected in crude conditioned medium (FIG. 11B), which indicates that FLAG-EPSTI1 is not secreted by the transfected cells.

### EXAMPLE 8

### Preparation of Polyclonal Antiserum

EPSTI1 was amplified using Expand™ High Fidelity PCR System (Boehringer Mannheim, purchased from Ercopharm Roche, Hvidovre, Denmark) with the primers:

| | |
|---|---|
| Fw | 5′-TTGGAGAATTCCATGAACACCCGCAATAGA-3′ |
| and | |
| Rv | 5′-AGGAAGCTTCCATATACCCCAGCTGTTACCGCT-3′ |

and the following PCR conditions: 4 min at 94° C., 28 cycles of 1 min at 94° C., 1 min at 56° C. and 1 min at 72° C., extension for 7 min at 72° C. and hold at 4° C. After amplification the fragment was inserted into the pET-28b+ vector (Novagen, purchased from Bie & Berntsen, Rødovre, Denmark) between the Eco RI and Hind III sites. 5 ng of this preparation was used to transform the E. coli strain Rosetta™ (DE3)pLysS Competent cells (Novagen, purchased from Bie & Berntsen, Rødovre, Denmark). LB medium (1 litre contains 10 g tryptone, 5 g yeast extract, 5 g NaCl, 1 ml 1M NaOH) containing 30 μg/ml kanamycin (Sigma-Aldrich, Vallensbaek, Denmark) was inoculated with a bacterial colony harboring the expression plasmid og grown overnight at 37° C. in a shaking incubator. The overnight culture was diluted 1:100 in 250 ml fresh LB medium with kanamycin and grown until the OD₆₀₀ reached 0.6. IPTG (Isopropyl β-_{D}-thiogalactoside, Sigma-Aldrich, Vallensbaek, Denmark) was added to a final concentration of 1 mM of the culture and grown for 3 hours. The cells were harvested by centrifugation at 3,500×g for 10 min. The His-tagged Epstil was purified using B-PER™ 6×His Fusion Protein Purification Kit (Pierce, purchased from Bie & Bemtsen, Rødovre, Denmark) with modifications. The cells were resuspended in 10 ml B-PERT Reagent, gently shaken at room temperature for 10 min and spinned at 18,500×g for 30 min. The supernatant was removed and the extraction repeated. The supernatant was diluted 1:1 in 50 mM NaH₂PO₄ and 300 mM NaCl (Merck, Albertslund, Denmark), pH 7.5 and spanned at 18,500×g for 30 min. The rest of the purification was performed according to the manufacturer's instructions with three elutions.

Two BALB/c mice were injected subcutaneously with about 8 μg of purified antigen (His-tagged Epstil1) every two weeks for six weeks. One week after the last injection, tail bleeds were performed to obtain antiserum containing polyclonal antibodies. The antisera were tested with ELISA.

### Example 9

### ELISA

Materials and Methods

ELISA plates were coated overnight at 4° C. on a shaker with antigen (purified His-tagged Epsti1), diluted in coating buffer (0.05 M carbonat-bicarbonat buffer, 0.016 M Na₂CO₃, 0.034 M NaHCO₃ pH 9.6 (Merck, Albertslund, Denmark)). After washing in PBS (phosphate-buffered saline, pH 7.2) the plates were blocked with 0.5% BSA (bovine serum albumin, fraction V, Sigma, Vallenbaek, Denmark) in PBS for 30 min. The plates were washed in PBS with 0.1% Twee®20 pH 7.2 (Merck, Albertslund, Denmark) before Incubating overnight at 4° C. on a shaker with the primary antibodies (the antisera). Superfluous primary antibodies were washed away with PBS— Twee®20 and the secondary antibody was added (HRP-conjugated rabbit a mouse, P0161, Dako, Glostrup, Denmark, diluted 1:2000 in PBS-Twee®20) and Incubated for 1 hour on a shaker. The plates were washed with PBS-Tween®20 and 100 μl OPD (o-Phenylenediamine, Sigma-Aldrich, Vallensbaek, Denmark) with H₂O₂ was added (4 tablets in 12 ml mQ H₂O with 5 μl 30% H₂O₂ (Merck, Albertslund, Denmark). When a yellow colour started to develop in some of the wells, the reaction was stopped with 0.5 M H₂SO₄ (Merck, Albertslund, Denmark) and the plate was read on Sunrise Absorbance Reader (Tecan Austria GmbH purchased from Laboratory, Automation & Technology A/S, Valby, Denmark) with XREAD PLUS Version V4.04.

Results

Both epsti1 and a random His-tagged protein were tested with the antiserum. An anti-His-antibody was included as a positive control and serum from a non-immunized mouse was used as a negative control. The polyclonal antiserum at a 1:200 dilution from one of the mice specifically recognized His-Epstil at a concentration of approximately 2 μg/ml whereas it did not recognize the random His-tagged protein.

TABLE 3
Sequence list.

| | | |
|---|---|---|
| seq | | |
| id | sequence in the | |
| no | 5′→3′ direction | description |

| | | |
|---|---|---|
| 1 | see FIG. 2A | bresi/epsti 1 cDNA |
| 2 | see FIG. 2B | bresi/epsti 1 protein |
| | | sequence |
| 3 | ACGACTCACTATAGGGCTTTTTTTTT | 12 oligo(dT) anchored |
| | TTTXX | T7 3′ primers |
| 4 | ACAATTTCACACAGGAXXXXXXXXXX | arbitrary M13r 5′ |
| | | primers |
| 5 | GTAATACGACTCACTATAGGGC | T7 promoter 22-mer |
| | | primer |
| 6 | AGCGGATAACAATTTCACACAGGA | full-length M13 |
| | | reverse (−48) 24-mer |
| | | primer |
| 7 | GAAGGTGAAGGTCGGAGT | GAPDH primer for |
| | | real-time RT-PCR |
| 8 | GAAGATGGTGATGGGATTTC | GAPDH primer for |
| | | real- time RT-PCR |
| 9 | GGCACCACTCCACTGTATCC | Tata box binding |
| | | protein primer for |
| | | real-time RT-PCR |
| 10 | GCACACCATTTTCCCAGAAC | Tata box binding |
| | | protein primer for |
| | | real-time RT-PCR |
| 11 | CGAAAACACCCTGCAATCTT | Vimentin primer for |
| | | real-time RT-PCR |
| 12 | TTGGCAGCCACACTTTCATA | Vimentin primer for |
| | | real-time RT-PCR |
| 13 | AGCATCGCTCTCCTGCTAAC | Thy-1 primer for |
| | | real-time RT-PCR |
| 14 | GCACGTGCTTCTTTGTCTCA | Thy-1 primer for |
| | | real-time RT-PCR |
| 15 | GAGGTGGATTCCGCTCCGGGCA | Cytokeratin 19 primer |
| | | for real-time RT-PCR |
| 16 | ATCTTCCTGTCCCTCGAGCAG | Cytokeratin 19 primer |
| | | for real-time RT-PCR |
| 17 | CTCTACTGCCAGGAAATGC | EPSTI1 primer for |
| | | real-time RT-PCR |
| 18 | GCCTGTAGCAGGATAGCTC | EPSTI1 primer for |
| | | real-time RT-PCR |
| 19 | CTTTTTGCAGAGGCCAATA | Adrenal gland protein |
| | | primer for real-time |
| | | RT-PCR |
| 20 | GTGCGACCGACTGGAATAAC | Adrenal gland protein |
| | | primer for real-time |
| | | RT-PCR |
| 21 | GTAGGGATTAAAATCTAAAA | gene specific primer |
| | | GSP-1 |
| 22 | GGTCAAGTGTGTGGGCAGTTG | gene specific primer |
| | | GSP-2 |
| 23 | CCAACAGCCTCCAGATTGCT | gene specific primer |
| | | GSP-3 |
| 24 | CCCAGCTGTTACCGCTATTCA | gene specific primer |
| | | GSP-4 |
| 25 | GCTGCCGTTTCAGTTCCAGT | gene specific primer |
| | | GSP-5 |
| 26 | GGTGAACCGGTTTAGCTCTG | gene specific primer |
| | | GSP-6 |
| 27 | CTTCCACTTCTCCAGGTTGG | gene specific primer |
| | | GSP-7 |
| 28 | TTAGGGGCTGCCTCCAAAC | gene specific primer |
| | | GSP-8 |
| 29 | CAGGAGTGACTGGCTTCTCC | human specific EPSTI1 |
| | | primer |
| 30 | AAGACCCCCAAAGCTTTCAA | human specific EPSTI1 |
| | | primer |
| 31 | ATGAACACCCGCAATAGAGTG | EPSTI1 primer for |
| | | RT-PCR across the |
| | | entire ORF |
| 32 | CGGTCGACGCCACCATGAACACCCGC | Forward primer for |
| | AATAGA | FLAG tagging of |
| | | EPSTI1 |
| 33 | CCATCGATGGTCACTTGTCATCGTCG | reverse primer for |
| | TCCTTGTAGTCTATACCCCAGCTGTT | FLAG tagging of |
| | ACC | EPSTI1 |
| 34 | MetAsnThrArgAsnArgValVal | EPSTI1 polypeptide |
| | AsnSerGlyLeuGlyAlaSerPro | fragment |
| | AlaSerArgProThrArgAspPro | |
| | GlnAspProSerGlyArgGlnGly | |
| | GluLeuSerProValGluAspGln | |
| | ArgGluGlyLeuAlaAlaProLys | |
| | GlyProSerArgGluSerValVal | |
| | HisAlaGlyGlnArgArgThrSer | |
| | AlaTyrThrLeuIleAla | |

### FIGURE LEGENDS

FIG. 1. Differential display of RNA profiles of tumour cells and fibroblasts cultured in separate- of co-cultures in a 3-dimensional tumour environment assay leads to identification of genes which are switched on or off during epithellal-stromal interaction. A, Phase contrast micrographs of MCF-7 and fibroblasts cultured in separate- or in co-culture, which leads to extensive interaction. B, Differential display of RNA extracted from a co-culture (c) versus RNA mixed from MCF-7 and fibroblasts cultured separately (s) by use of four different primer combinations and run as duplicate samples. Two amplicons of differential abundance of approximately 600 bp appear in lane 9-12 (box) as obtained with primers AP9/ARP1 (HIEROGLYPH). C, Differential expression was verified by real-time PCR as relative gene expression using gene-specific primers and normalisation with two house-keeping genes (GAPDH, TATA box binding protein (TBP)) and lineage-specific markers (vimentin and Thy-1 for fibroblasts and cytokeratin 19 for MCF-7 cells). D, Using gene-specific primers and real-time PCR, differential expression of the 580-bp transcript in co-culture (black bar) versus separate culture (shaded bar) was verified and compared to a non-differentially expressed amplicon, identified as adrenal gland protein (AGP).

FIG. 2. A, EPSTI1 5′RACE generated of full-length cDNA consensus sequence of 1508 bp in FASTA format. cDNAs generated from normal breast tissue and placenta tissue were identical to the consensus sequence. Start (ATG) and stop codons (TGA) are shaded. B, The open reading frame of EPSTI1 encodes a protein with the sequence of 307 aa.

FIG. 3. EPSTI1 maps to chromosome 13 q and contains 11 exons spanning a 104.2 kb region with a start codon in exon 1 and a stop codon in exon 11.

FIG. 4. The relative expression of EPSTI1 using real time PCR. A, Samples of normal breast (reference samples: a-e, range 0.82-1.2) were compared to samples of invasive breast carcinomas (f-m, range 2.5-65). B, EPSTI1 expression in a tissue panel compared to normal breast. 1: normal breast (reference sample), 2: normal breast, 3: prostate, 4: testis; 5: skeletal muscle, 6: uterus, 7: placenta, 8: adrenal gland, 9: pancreas, 10: salivary gland, 11: foetal brain, 12: medullary cord, 13: brain, 14: colon, 15: heart, 16: bone marrow, 17: kidney, 18: small intestine, 19: liver, 20: spleen, 21: lung, 22: stomach, 23: trachea, 24: thymus, 25: foetal liver.

FIG. 5. Chromosomal localization of the EPSTI1 gene. Using human specific EPSTI1 primers and human monochromosomal somatic cell hybrids, EPSTI1 is localized to chromosome 13 q. Dotted line indicates localization mapped in silico to 13q13.3. DNA samples include chromosome 13 (NA11689), fragments of chromosome 13 (NA11766, NA11767, NA14050, NA11575), and chromosome 12 (NA10868) as a negative control and breast cDNA (cDNA) as a positive control. Solid bars indicate part of chromosome 13 contained in the somatic cell hybrids. The lower panel shows result of the PCR performed with human specific EPSTI1 primers.

FIG. 6. Full-length cDNA and predicted amino acid sequence of the EPSTIL gene. Nucleotides (open reading frame in capital letters) and amino acids (in single-letter code) are numbered to the left. Nucleotides representing the putative translation initiation codon (ATG) and the stop codon (TGA) are shaded. The polyadenylation signal (MTAAA) is boxed and the poly(A) tail is underlined. Three possible coiled-coil domains of the predicted amino acid sequence are In boldface type. Intron-exon boundaries are indicated by brackets.

FIG. 7. Northern blot hybridisation and RT-PCR across the entire ORF of EPSTI1. (A) A commercial multiple tissue Northern blot was probed under high stringency and reveals an EPSTI1 transcript of approximately 1.5 kb in all tissues tested. Note the relative strong expression in placenta. (B) RT-PCR across the entire ORF confirms the transcript size of 1.5 kb (left lane, marker), and reveals no signs of alternative splicing of EPSTI1 in placenta (middle lane) or breast carcinoma (right lane).

FIG. 8. Alignment of the predicted amino acid sequences of EPSTI1 and the mouse homolog, BAB30623. (A) BAB30623 overlaps 219 out of 307 amino acids of EPSTI1, and the sequences display 64% identity and 77% similarity, respectively. Identical amino acids are shaded black, and similar (i.e. amino acids are shaded grey. (B) Comparison of the overhanging C-terminal 88 amino acids of EPSTI1 with the preceding 219 amino acids and BAB30623, identifies a possible repeat sequence (position 230-262) in EPSTI1.

FIG. 9. Overexpression of EPSTI1 in breast cancer and expression profile in other tissues as assessed by real-time PCR. (A) Samples of normal breast (N-1-N5, range 0.7-1.5, first bar: reference sample) compared to samples of invasive breast carcinomas, which all overexpress EPSTI1 (T1-T8, range 5.6-72.1). (B) EPSTIL expression in a tissue panel compared to normal breast (reference). The expression of EPSTI1 is most prominent in placenta.

Error bars represent standard deviation of triplicate samples.

FIG. 10. EPSTIL is expressed primarily in the epithelial compartment as assessed by real-time PCR.

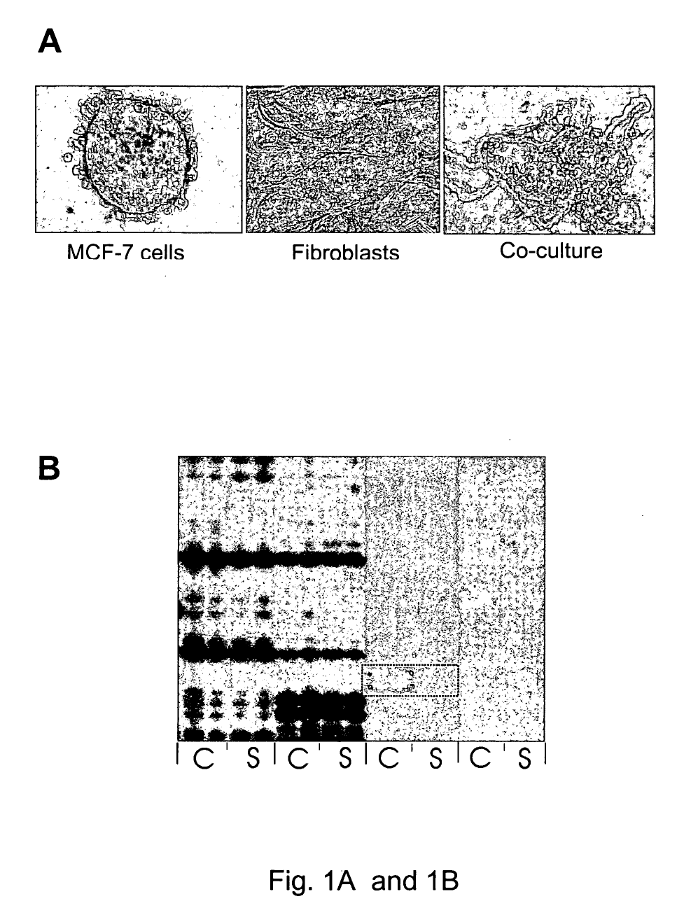

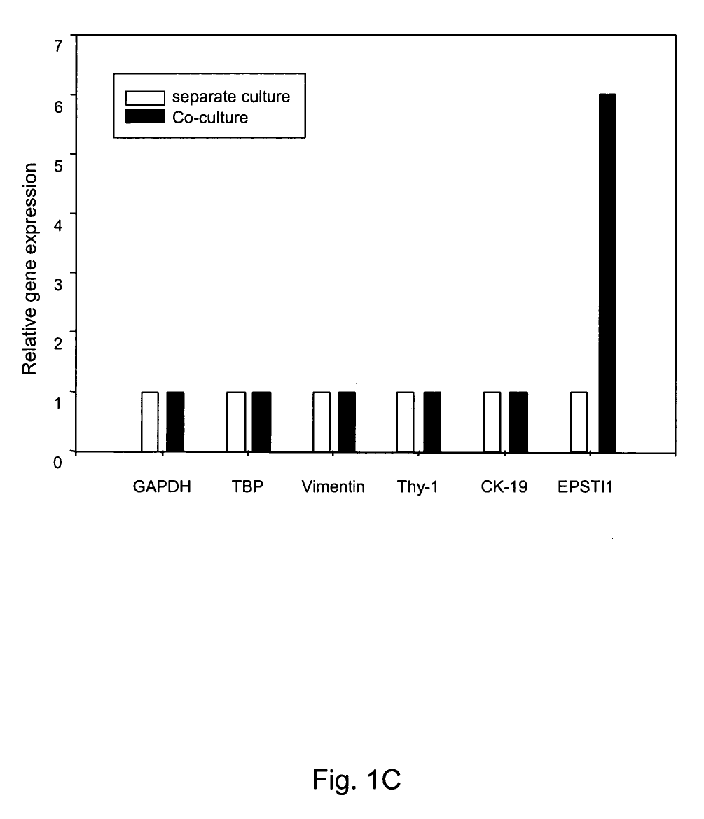

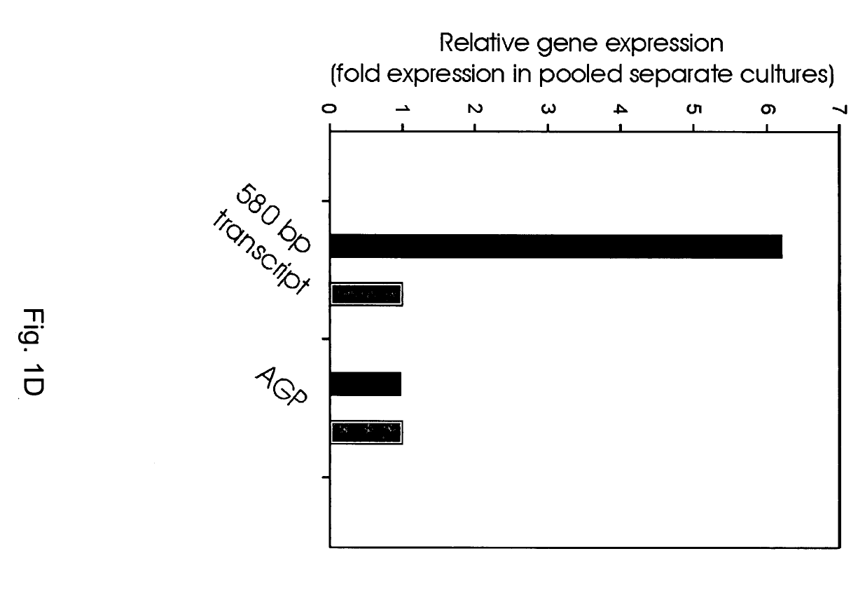

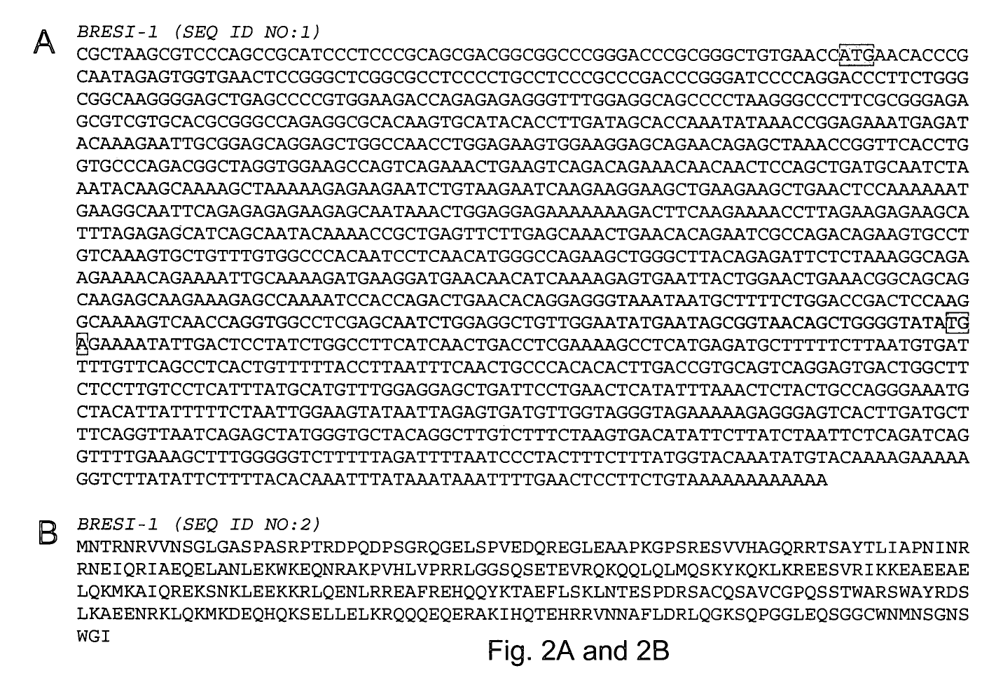

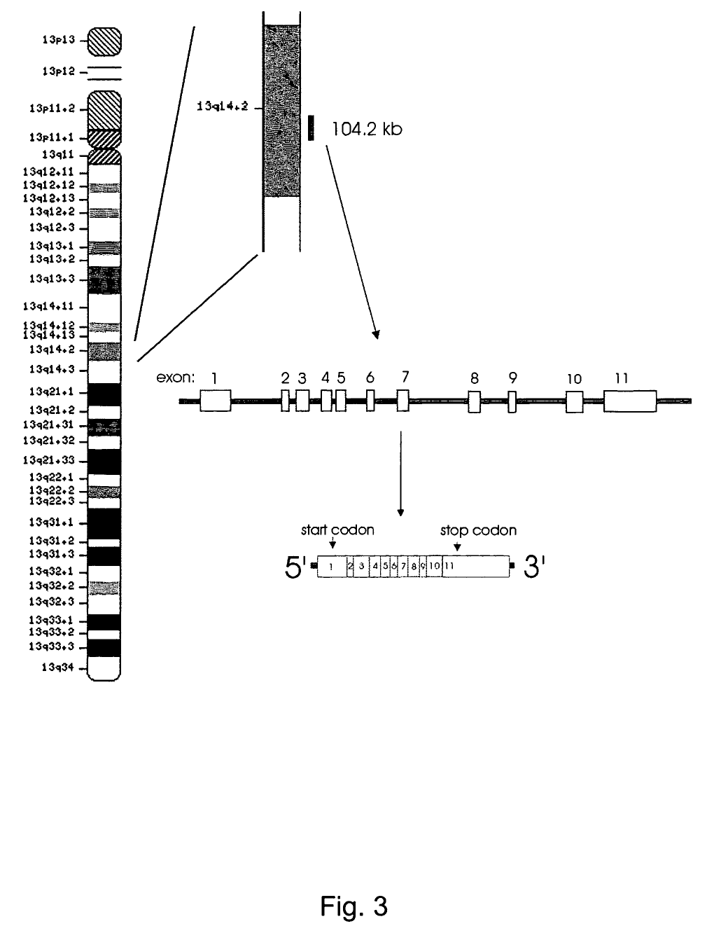

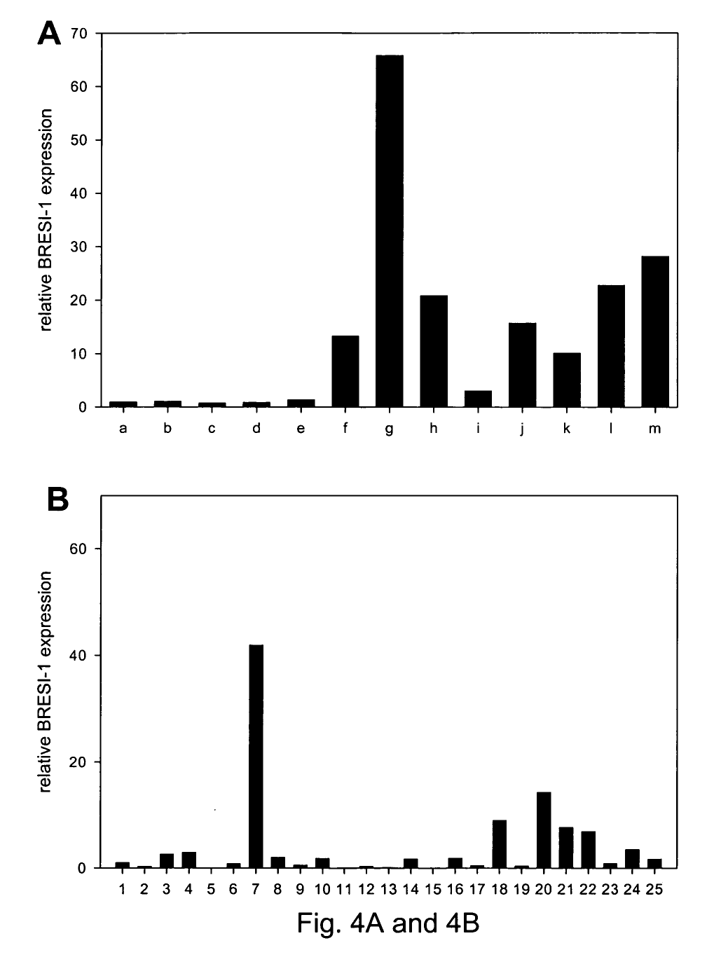

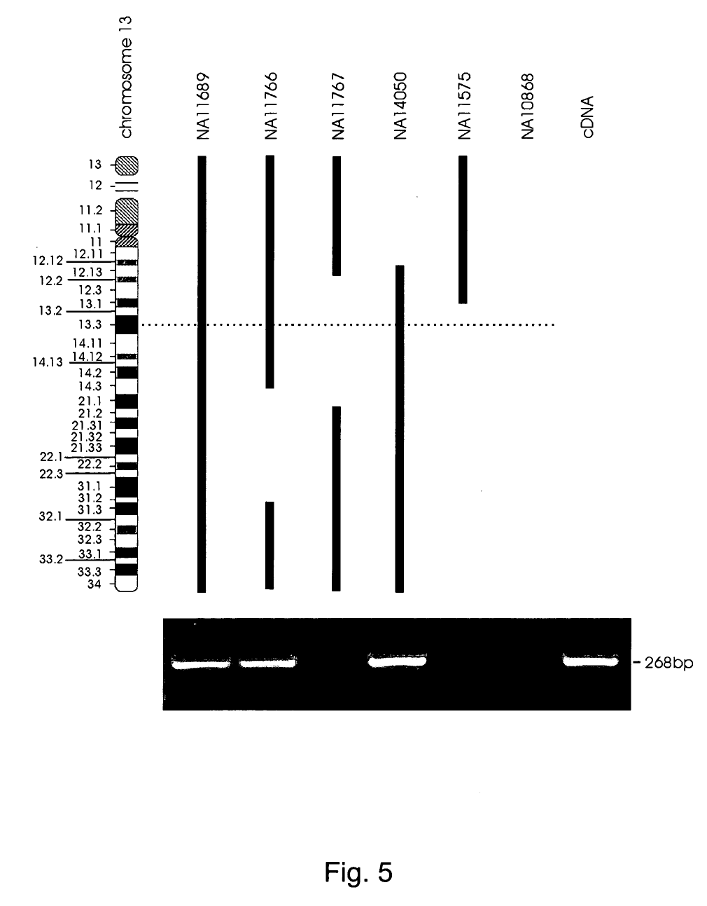

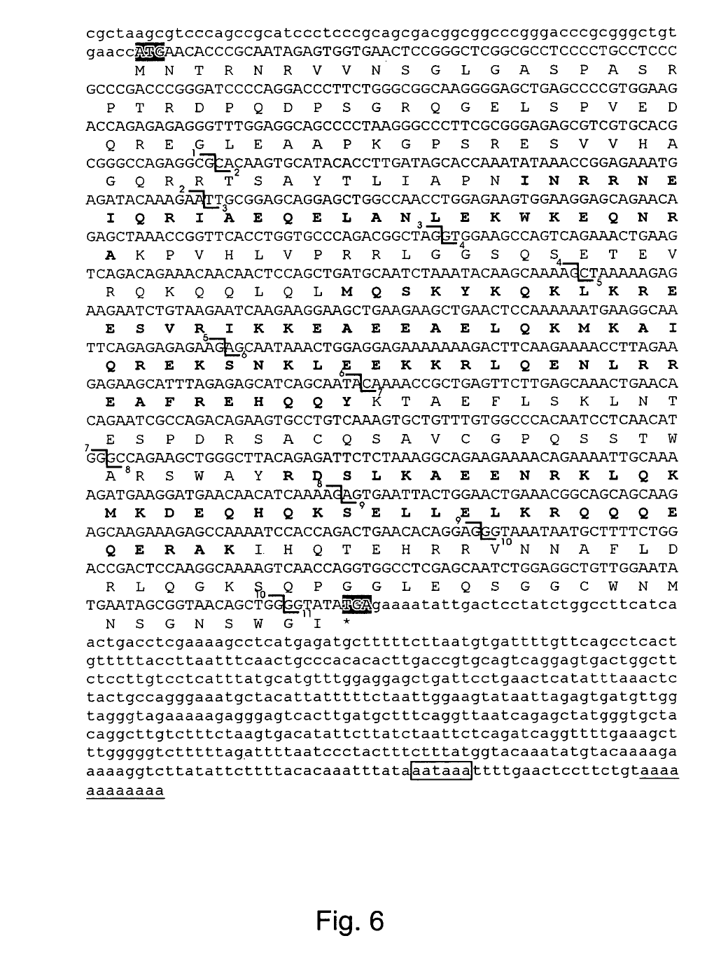

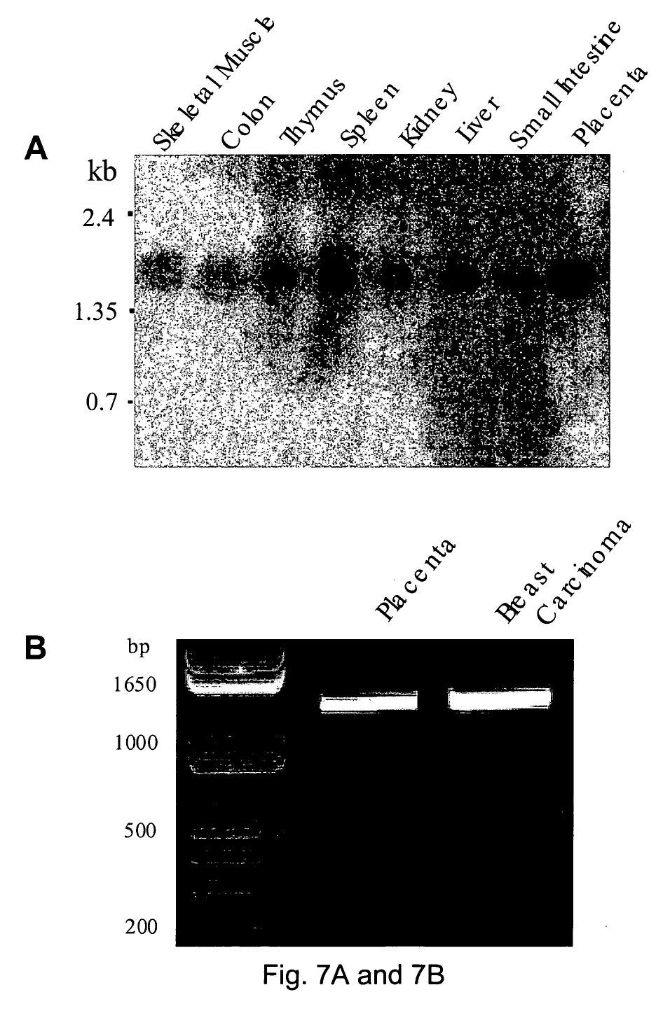

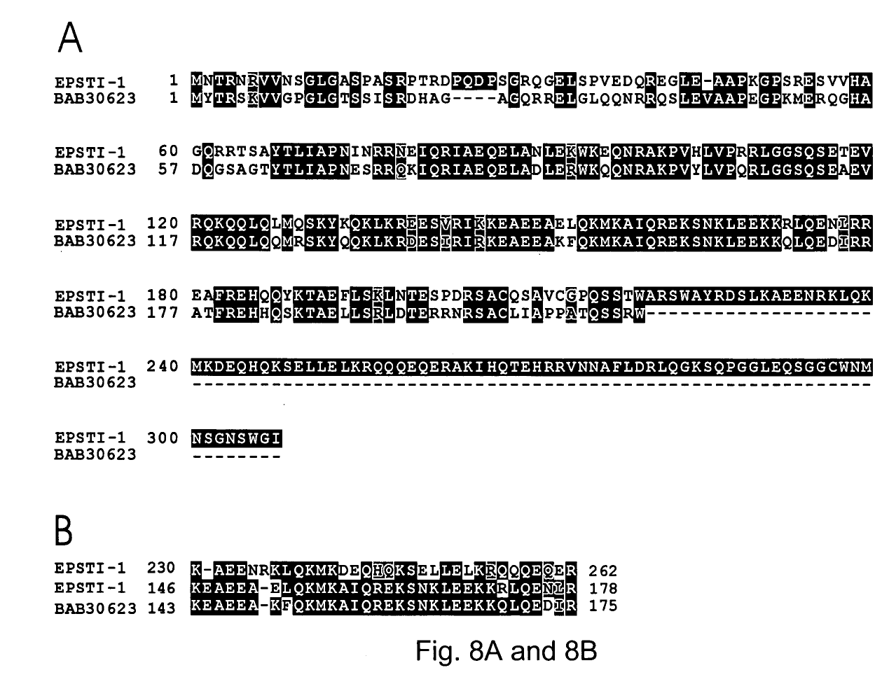

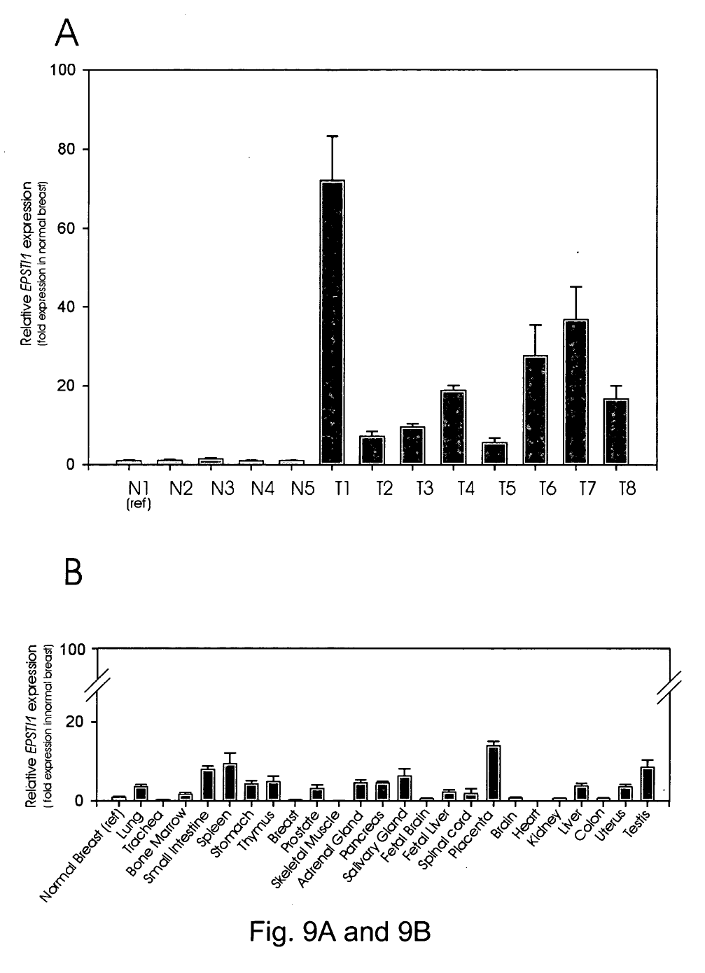

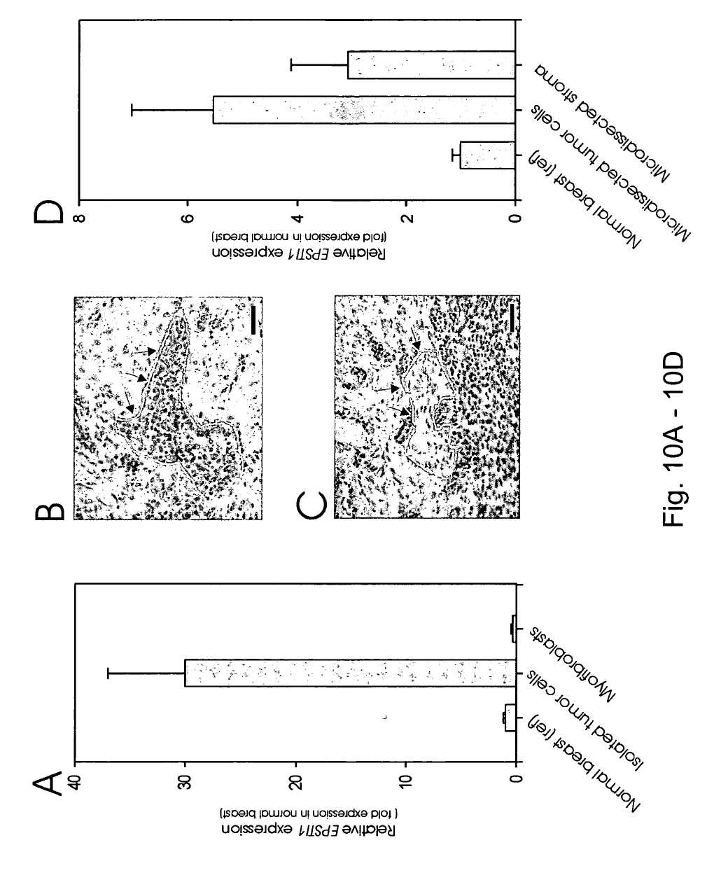

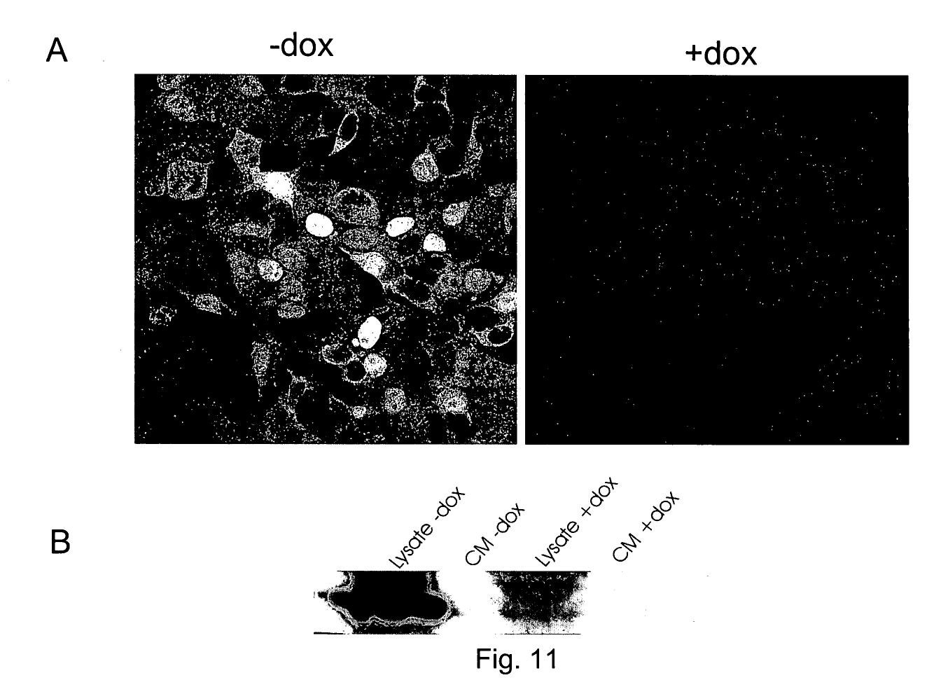

(A) A sample of normal breast (1.0, reference sample, ref) compared to samples of isolated tumour cells (30.0) or experimentally generated myofibroblast (0.3), of which only tumour cells exhibit upregulation of EPSTI1. Error bars represent standard deviation of triplicate samples.

(B and C) Cryosections of an invasive breast carcinoma counter-stained with hematoxylin prior to laser-assisted microdissection. (B) Tumour cells and (C) stroma, respectively, were microdissected and collected by laser pressure catapulting. Arrows indicate the photolysed separation area.

(D) Microdissected tumour cells exhibit a higher relative expression level of EPSTI1 (5.5) than microdissected stroma (3.1), whereas both tumour compartments display upregulation of EPSTI1 as compared to the normal breast reference (1.0, ref). Error bars represent standard deviation of triplicate samples. bar: 200 μm.

FIG. 11. (A) Conditional expression of FLAG-tagged EPSTI1 locates expression to both the nucleus and the cytoplasm. Immunocytochemical analysis of the MCF7 FLAG-EPSTIL cell line demonstrates FLAG-tagged EPSTI1 conditional expression in either the nucleus or the cytoplasm, or in both compartments (left, −dox) as compared to no expression in the presence of deoxycycline in the medium (right, +dox). (B) That FLAG-tagged EPSTI1 protein was indeed regulated by dox was confirmed by Western blot analysis. Lysate of cells without dox contained FLAG-tagged protein, whereas in lysate of cells with dox and conditioned media FLAG-tagged protein could not be detected.

### REFERENCES

Ahmad, A., Hanby, A., Dublin, E., Poulsom, R., Smith, P., Bames, D., Rubens, R., Anglard, P. and Hart, I. (1998). Stromelysin 3: An independent prognostic factor for relapse-free survival in node-positive breast cancer and demonstration of novel breast carcinoma cell expression. Am. J. Pathol. 152, 721-728.

Altschul et al. (1997) Nucleic Acids Res. 25:3389-402

Altschul, et al. (1990) J. Mol. Biol. 215:403-410

Ausubel et al. (2000) Current protocols in molecular biology, John Wiley and Sons, Inc.

Basset, P., Bellocq, J. P., Wolf, C., Stoll, I., Hutin, P., Limacher, J. M., Podhajcer, 0. L., Chenard, M. P., R10, M. C. and Chambon, P. (1990). A novel metalloproteinase gene specifically expressed in stromal cells of breast carcinomas. Nature 348, 699-704.

Basset, P., Wolf, C. and Chambon, P. (1993). Expression of the stromelysin-3 gene in fibroblastic cells of invasive carcinomas of the breast and other human tissues: a review. Breast Cancer Res. Treat. 24, 185-193.

Boyd, R. S. and Balkwill, F. R. (1999). MMP-2 release and activation in ovarian carcinoma: The role of fibroblasts. Br. J. Cancer 80, 315-321.

Brenner et al. (1998) Proc. Natl. Acad. Sci. USA 95: 6073-6078

Bustin, S. A. (2000). Absolute quantification of mRNA using real-time reverse transcription polymerase chain reaction assays. J. Mol. Endocrinol. 25, 169-193. Cole, et al., 1985, in Monoclonal Antibodies and Cancer Therapy, Alan R. Liss, Inc., pp. 77-96.

Drwinga et al. (1993) Genomics 16: 311-314

Elenbaas, B. and Weinberg, R. A. (2001). Heterotypic signalling between epithelial tumour cells and fibroblasts in carcinoma formation. Exp. Cell Res. 264, 169-184.

Encyclopedia of Life Sciences/www.els.net, Nature Publishing Group, (2000)

Engel, G., Heselmeyer, K., Auer, G., Backdahl, M., Eriksson, E. and Linder, S. (1994). Correlation between stromelysin-3 mRNA level and outcome of human breast cancer. Int. J. Cancer 58, 830-835.

Harlow, et al. (Antibodies: A Laboratory Manual, Cold Spring Harbor Press, 1988)

Kawaguchi et al. (1998) Proc Natl Acad Sci USA 95:1062-1066.

Karlin and Altschul (1990) Proc. Natl. Acad. Sci. USA 87:2264-2268.

Karlin and Altschul (1993) Proc. Natl. Acad. Sci. USA 90:5873-5877.

Kohler and Milstein, (1975), Nature, 256:495-497

Kozbor et al., (1983) Immunology Today 4:72

Lockhart et al., Nature Biotechnology (1996) 14: 1675-1680

Liang P, Pardee AB. (1992) Differential display of eukaryotic messenger RNA by means of the polymerase chain reaction. Science 257:967-71.

Liotta, L. A. and Kohn, E. C. (2001). The microenvironment of the tumour-host interface. Nature 411, 375-379.

Madden et al. (1996) Meth. Enzymol. 266:131-141;

Maruyama, K. and Sugano, S. (1994). Oligo-capping: A simple method to replace the cap structure of eukaryotic mRNAs with oligoribonucleotides. Gene 138, 171-174.

Morales, C. P., et al. (1999) Nature Genetics 21: 115-118.

Pearson W. R and D. J. Lipman (1998)Proc Natl Acad Sci USA 85:2444-2448,

Péchoux, C., Gudjonsson, T., Rønnov-Jessen, L., Bissell, M. J. and Petersen, O. W. (1999). Human mammary luminal epithelial cells contain progenitors to myoepithelial cells. Dev. Biol. 206, 88-99.

Quackenbush et al. (2000) Nucleic Acids Res. 28: 141-145

Radisky, D., Hagios, C. and Bissell, M. J. (2001). Tumours are unique organs defined by abnormal signaling and context. Cancer Biol. 11, 87-95.

Rønnov-Jessen, L. and Petersen, 0. W. (1993). Induction of a-smooth muscle actin by transforming growth factor-bl in quiescent human breast gland fibroblasts. Implications for myofibroblast generation in breast neoplasia. Lab. Invest. 68, 696-707.

Rønnov-Jessen, L., Petersen, O. W. and Bissell, M. 3. (1996). Cellular changes involved in conversion of normal to malignant breast: The importance of the stromal reaction. Physiol. Rev. 76, 69-125.

Rønnov-Jessen, L., Petersen, O. W., Kotellansky, V. E. and Bissell, M. J. (1995). The origin of the myofibroblasts in breast cancer: Recapitulation of tumour environment in culture unravels diversity and implicates converted fibroblasts and recruited smooth muscle cells. J. Clin. Invest. 95, 859-873.

Rønnov-Jessen, L., van Deurs, B., Cells, J. E. and Petersen, 0. W. (1990). Smooth muscle differentiation in cultured human breast gland stromal cells. Lab. Invest. 63, 532-543.

Rønnov-Jessen, L., van Deurs, B., Nielsen, M. and Petersen, O. W. (1992). Identification, paracrine generation and possible function of human breast carcinoma myofibroblasts in culture. In Vitro Cell. Dev. Biol. 28A, 273-283.

Sambrook et al, (1989) Molecular Cloning: A Laboratory Manual, Second Edition, Cold Spring Harbor, N.Y.,

Schnack Nielsen, B., Sehested, M., Timshel, S., Pyke, C. and Danø, K. (1995). Messenger RNA for urokinase plasminogen activator (uPA) is expressed in myofibroblasts adjacent to cancer cells in human breast cancer. Lab. Invest. 74, 168-177.

Schultz et al. (2000) Nucleic Acids Res. 28: 231-234; http://smart.embl-heidelberg.de

Shena et al., Science (1995) 270: 467-470

Steve Rozen, Helen 1. Skaletsky (1998) Primer3. http://www-genome.wi.mit.edu/cgi-bin/primer/primer3_www.cgi; Code available at http://www-genome.wi.mit.edu/genome_software/other/primer3.html

Tisty, T. D. and Hein, P. W. (2001). Know thy neighbor: stromal cells can contribute oncogenic signals. Curr. Op. Genet. Dev. 11, 54-59.

Zhang, J. & Madden, T. L. (1997) Genome Res. 7:649-656.

1. An isolated nucleic acid molecule encoding a polypeptide selected from the group consisting of:
a) the polypeptide EPSTI1 set forth in SEQ ID NO:2; and
b) a polypeptide comprising a fragment of SEQ ID NO: 2 comprising at least 9 consecutive amino acids of SEQ ID NO: 34.

2. An isolated nucleic acid molecule having the nucleic acid sequence of SEQ ID NO:1 encoding the EPSTI1 polypeptide;

3. A nucleic acid sequence which is complementary to any of the nucleic acid sequences selected from the group consisting of the nucleic acid sequences according to claim 1.

4. A cDNA sequence according to claim 1.

5. A genomic DNA sequence consisting of a nucleotide sequence according to claim 1.

6. A double stranded nucleic acid sequence according claim 1.

7. A single stranded nucleic acid sequence according to claim 1.

8. The nucleic acid according to claim 1, wherein the encoded polypeptide is of mammalian origin.

9. The nucleic acid according to claim 1, wherein the encoded polypeptide is of human origin.

10. The nucleic acid molecule according to claim 1, wherein said nucleotide sequence comprises a heterologous nucleotide sequence.

11. The nucleic acid molecule according to claim 1, wherein said heterologous nucleotide sequence encodes a heterologous polypeptide.

12. An oligonucleotide capable of hybridising to a nucleic acid according to claim 1 for use as a medicament.

13. A method for making a recombinant vector comprising inserting the nucleic acid molecule according to claim 1 into a vector.

14. A recombinant vector comprising the nucleic acid molecule according to claim 1.

15. The recombinant vector according to claim 14, wherein said nucleic acid molecule is operably linked to a heterologous regulatory sequence that controls gene expression.

16. A recombinant host cell comprising the nucleic acid molecule according to claim 13.

17. An isolated polypeptide comprising an amino acid sequence selected from the group consisting of:
a) an amino acid sequence of SEQ ID NO:2;
b) an amino acid sequence having at least 74% homology compared to the total number of positions in the sequence of SEQ ID NO:2.

18. An isolated polypeptide comprising an amino acid sequence comprising a fragment of SEQ ID NO: 2 comprising at least 9 consecutive amino acids of SEQ ID NO: 34.

19. A polypeptide or polypeptide fragment according to claim 17 which is substantially purified.

20. A polypeptide or polypeptide fragment according to claim 17, wherein the polypeptide or polypeptide fragment has been modified compared only by conservative substitutions.

21. A fusion polypeptide comprising at least one polypeptide fragment according to claim 17 and at least one fusion partner, said fusion partner being selected from the group consisting of GFP, GST, Myc, HIS, Flag and V5.

22. A polypeptide or polypeptide fragment according to claim 17 coupled to a carbohydrate or a lipid moiety.

23. A polypeptide according to claim 17 which is glycosylated and/or phosphorylated.

24. A substantially pure polypeptide according to claim 17 for use as a medicament.

25. A method for producing a polypeptide according to claim 17, comprising:
(a) culturing a host cell according to claim 16 under conditions suitable to produce a polypeptide encoded by the nucleic acid molecule of claim 1; and
(b) recovering the polypeptide from the cell culture.

26. A purified antibody or antibody fragment which specifically binds to the polypeptide according to claim 18.

27. An antibody according to claim 26 which is a polyclonal antibody.

28. An antibody according to claim 26 which is a monoclonal antibody.

29. A method for determining the presence of a EPSTI1 protein in a sample comprising the steps:
a) contacting a sample or preparation thereof with an antibody or antibody fragment according to claim 26 which selectively binds the EPSTI1 polypeptide; and
b) detecting whether said EPSTI1 polypeptide is bound by said antibody and thereby detecting the EPSTI1 polypeptide.

30. The method according to claim 29, wherein said antibody, or said antibody fragment, is labelled.

31. The method according to claim 30, wherein the label is selected from the group consisting of, radioisotopes, fluorescent compounds, enzymes, (electro)chemoluminescent compounds or a member of an affinity pair.

32. The method according to claim 29, wherein the method is used in an immunohistochemical assay to detect or quantify the presence of EPSTI1 in a sample.

33. The method according to claim 29, wherein the method is used in a in vitro ELISA assay to detect or quantify the presence of EPSTI1 in a sample.

34. A method for determining the presence of EPSTI1 mRNA is present in a sample, the method comprising:
a) obtaining a sample comprising mRNA from a test subject;
b) contacting the test sample with an isolated nucleic acid molecule that hybridizes under conditions of hybridisation to the EPSTI1 mRNA; and
c) determining that the EPSTI1 mRNA is present in the sample when the sample contains mRNA that selectively hybridizes to the isolated nucleic acid molecule;
wherein the EPSTI1 mRNA is selected from the group consisting of:
d) a mRNA molecule that encodes a polypeptide comprising the amino acid sequence of SEQ ID NO:2;
e) a mRNA molecule corresponding to the nucleic acid sequence SEQ ID NO:1; or the complement thereof;

35. A method for determining the relative level of EPSTI1 mRNA in a sample, the method comprising:
a) obtaining a sample comprising mRNA from a test subject and from a control subject;
b) contacting the test sample the control sample with at least one nucleic acid molecule that hybridizes under conditions of hybridisation to the EPSTI1 mRNA; and
c) determining the realtive level of the EPSTI1 mRNA in the test sample by comparing the EPSTI1 mRNA specific signal in the test sample to the signal in the control sample.
wherein the EPSTI1 mRNA is selected from the group consisting of:
d) a mRNA molecule that encodes a polypeptide comprising the amino acid sequence of SEQ ID NO:2;
e) a mRNA molecule corresponding to the nucleic acid sequence SEQ ID NO:1; or the complement thereof;

36. A method according to claim 29, wherein the method is performed on a sample comprising an extract from a cancer tissue or a suspected cancer tissue.

37. The method of claim 29, wherein the sample is isolated from tissues selected from the group of tissues consisting of breast, placenta, lymphoid tissue, ovary, testis, thymus, lung, stomach, small intestine, colon, pancreas, stomach, spleen, skin and extracellular body fluids.

38. The method of claim 37, wherein the presence of detectable EPSTI1 polypeptide or mRNA in the test sample indicates that the test subject has or is at risk of developing metastatic cancer.

39. The method of claim 38, wherein the metastatic cancer is selected from the group consisting of breast cancer, cancer of the male and female genital tract, and cancer of the thymus, lung, lymphoid tissue, stomach, small intestine, prostate, adrenal gland, pancreas, colon, pancreas, liver, salivary gland, spleen and skin.

40. A method for determining whether an individual has at least an increased likelihood of metastatic cancer comprising determining the presence of EPSTI1 expression in said tissue or tissue extract.

41. A method according to claim 40, wherein the determination of the EPSTI1 expression is performed by contacting tissue or tissue extracts of a mammal to be tested with an EPSTI1 nucleic acid probe, for a time and under conditions sufficient to allow hybridization of said probe with EPSTI1 mRNA expressed in said tissue or tissue extract and detecting said hybridization wherein said EPSTI1 mRNA is expressed in a tissue or tissue extracts from the individual.

42. The method according to claim 40, wherein said nucleic acid probe is DNA or RNA.

43. A method according to claim 40, wherein the determination of the EPSTI1 expression is performed by contacting tissue or tissue extracts of a mammal to be tested with an antibody or antigen binding fragment thereof which binds to EPSTI1 protein, for a time and under conditions sufficient to allow binding of said antibody or antigen binding fragment thereof to EPSTI1 protein in said tissue or tissue extract and detecting said binding wherein said EPSTI1 protein is present in said tissue or tissue extracts.

44. A method according to claim 40, wherein EPSTI1 expression is increased in said tissue or tissue extracts at least 10-fold compared to normal tissue or tissue extracts.

45. The method according to claim 29, wherein the method is used in a prognostic in vitro assay.

46. The method according to claim 29, wherein the method is used in a diagnostic in vitro assay.

47. A kit for detection of EPSTI1, comprising:
a) at least one first container adapted to contain a binding molecule which specifically binds EPSTI1 or a fragment of EPSTI1, said binding molecule being selected from the group consisting of an antibody which binds EPSTI1 or a fragment of EPSTI1, a nucleic acid fragment capable of binding to nucleic acid encoding EPSTI1 or a fragment of EPSTI1 and a compound capable of binding to EPSTI1 or a fragment of EPSTI1,
b) means for detecting binding, if any, or the level of binding, of the binding molecule to EPSTI1 or fragments of EPSTI1 or nucleic acids encoding EPSTI1.

48. A kit according to claim 47, wherein the binding molecule is labelled.

49. A kit according to claim 47 further comprising
c) directions for correlating whether binding, if any, or the level of binding, to said binding molecule is indicative of the individual mammal having a significantly higher likelihood of having metastatic cancer or a predisposition for having metastatic cancer.

50. A kit according to claim 47, wherein the nucleic acid fragment capable of binding to the nucleic acid encoding EPSTI1 or a fragment of EPSTI1 consists of at least one contiguous fragment of the human EPSTI1 gene of SEQ ID NO:1, wherein said fragment is at least 17 nucleotides in length.

51. A kit according to claim 47, wherein the antibody is an antibody which binds EPSTI1 of SEQ ID NO:2 or a fragment of EPSTI1.

52. A kit according to claim 51, wherein the antibody is a polyclonal antibody or a monoclonal antibody.

53. The kit according to claim 51, being an ELISA kit.

54. The kit according to claim 51 in which the antibody or antigen binding fragment thereof is packaged in an aqueous medium or in lyophilized form.

55. The kit according to claim 47 further comprising a second container adapted to contain reagents for detection of said mammal EPSTI1 expression.

56. The kit according to claim 47, wherein the kit is compartmentalised.

57. A method for isolation of nucleic acid sequences coded by genes which are regulated by the interaction between epithelial cells and the surrounding stroma cells, the method comprising:
a) extracting RNA from epthelial cells and stroma cells cultured as a co-culture in a three-dimensional culture system and from epithelial cells and stroma cells cultured as separate cultures in a similar three-dimensional culture system,
b) selecting two or more marker genes which are specific for the epithelial cell-lineage and the stroma cell-lineage, respectively,
c) determining the mRNA level of said cell-lineage specific markers in the RNA extracted from the co-culture as well as in the RNA extracted from the separate cultures of epithelial cells and stroma cells,
d) normalising the RNA extracted from the separate cultures by mixing (pooling) the RNA from the separate cultures to obtain ratios of the level of cell-lineage specific marker mRNAs that are similar to the ratios observed in the RNA isolated from the co-culture,
e) identifying transcripts or cDNA copies of transcripts which are differently representated in the RNA extracted from the co-culture relative to the normalised (pooled) RNA from separate cultures, and
f) isolating said transcripts and/or cDNA copies of transcripts.

58. A method according to claim 57 wherein the epithelial cells are cancer cells.

59. A method according to claim 57 wherein the stroma cells are fibroblasts.

60. A method according to claim 57, wherein the epithelial cell-lineage specific marker gene is cytokeratin 19 and the stroma cell-lineage marker genes are vimentin and thy-1.

61. A method according to claim 57 wherein the epithelial cells are breast cancer cells and the stroma cells are human telomerase (hTERT) transduced normal breast fibroblasts.

62. A polypeptide or polypeptide fragment according to claim 18 which is substantially purified.

63. A polypeptide or polypeptide fragment according to claim 18, wherein the polypeptide or polypeptide fragment has been modified compared only by conservative substitutions.

64. A fusion polypeptide comprising at least one polypeptide fragment according to claim 18 and at least one fusion partner, said fusion partner being selected from the group consisting of GFP, GST, Myc, HIS, Flag and V5.

65. A polypeptide or polypeptide fragment according to claim 18 coupled to a carbohydrate or a lipid moiety.

66. A polypeptide according to claim 18 which is glycosylated and/or phospho-rylated.

67. A substantially pure polypeptide according to claim 18 for use as a medicament.

68. A method for producing a polypeptide according to claim 18, comprising:
(a) culturing a host cell according to claim 16 under conditions suitable to produce a polypeptide encoded by the nucleic acid molecule of claim 1; and
(b) recovering the polypeptide from the cell culture.


SEQUENCE LISTING (33 sequences)

SEQ ID NO: 1 - DNA, 1508 bp, Homo sapiens
  CDS (66)...(989): EPSTI 1 coding region
cgctaagcgtcccagccgcatccctcccgcagcgacggcggcccgggacccgcgggctgtgaaccatgaacacccgcaatagagtggtgaactccgggctcggcgcctcccctgcctcccgcccgacccgggatccccaggacccttctgggcggcaaggggagctgagccccgtggaagaccagagagagggtttggaggcagcccctaagggcccttcgcgggagagcgtcgtgcacgcgggccagaggcgcacaagtgcatacaccttgatagcaccaaatataaaccggagaaatgagatacaaagaattgcggagcaggagctggccaacctggagaagtggaaggagcagaacagagctaaaccggttcacctggtgcccagacggctaggtggaagccagtcagaaactgaagtcagacagaaacaacaactccagctgatgcaatctaaatacaagcaaaagctaaaaagagaagaatctgtaagaatcaagaaggaagctgaagaagctgaactccaaaaaatgaaggcaattcagagagagaagagcaataaactggaggagaaaaaaagacttcaagaaaaccttagaagagaagcatttagagagcatcagcaatacaaaaccgctgagttcttgagcaaactgaacacagaatcgccagacagaagtgcctgtcaaagtgctgtttgtggcccacaatcctcaacatgggccagaagctgggcttacagagattctctaaaggcagaagaaaacagaaaattgcaaaagatgaaggatgaacaacatcaaaagagtgaattactggaactgaaacggcagcagcaagagcaagaaagagccaaaatccaccagactgaacacaggagggtaaataatgcttttctggaccgactccaaggcaaaagtcaaccaggtggcctcgagcaatctggaggctgttggaatatgaatagcggtaacagctggggtatatgagaaaatattgactcctatctggccttcatcaactgacctcgaaaagcctcatgagatgctttttcttaatgtgattttgttcagcctcactgtttttaccttaatttcaactgcccacacacttgaccgtgcagtcaggagtgactggcttctccttgtcctcatttatgcatgtttggaggagctgattcctgaactcatatttaaactctactgccagggaaatgctacattatttttctaattggaagtataattagagtgatgttggtagggtagaaaaagagggagtcacttgatgctttcaggttaatcagagctatgggtgctacaggcttgtctttctaagtgacatattcttatctaattctcagatcaggttttgaaagctttgggggtctttttagattttaatccctactttctttatggtacaaatatgtacaaaagaaaaaggtcttatattcttttacacaaatttataaataaattttgaactccttctgtaaaaaaaaaaaa

SEQ ID NO: 2 - protein, 307 aa, Homo sapiens
MNTRNRVVNSGLGASPASRPTRDPQDPSGRQGELSPVEDQREGLEAAPKGPSRESVVHAGQRRTSAYTLIAPNINRRNEIQRIAEQELANLEKWKEQNRAKPVHLVPRRLGGSQSETEVRQKQQLQLMQSKYKQKLKREESVRIKKEAEEAELQKMKAIQREKSNKLEEKKRLQENLRREAFREHQQYKTAEFLSKLNTESPDRSACQSAVCGPQSSTWARSWAYRDSLKAEENRKLQKMKDEQHQKSELLELKRQQQEQERAKIHQTEHRRVNNAFLDRLQGKSQPGGLEQSGGCWNMNSGNSWGI

SEQ ID NO: 3 - DNA, 31 bp, Artificial Sequence
  12 oligo(dT) anchored T7 3′ primers
acgactcactatagggcttttttttttttnn

SEQ ID NO: 4 - DNA, 26 bp, Artificial Sequence
  arbitrary M13r 5′ primers
acaatttcacacaggannnnnnnnnn

SEQ ID NO: 5 - DNA, 22 bp, Artificial Sequence
  T7 promoter 22-mer primer
gtaatacgactcactatagggc

SEQ ID NO: 6 - DNA, 24 bp, Artificial Sequence
  full-length M13 reverse (-48) 24-mer primer
agcggataacaatttcacacagga

SEQ ID NO: 7 - DNA, 18 bp, Artificial Sequence
  GAPDH primer for real-time RT-PCR
gaaggtgaaggtcggagt

SEQ ID NO: 8 - DNA, 20 bp, Artificial Sequence
  GAPDH primer for real-time RT-PCR
gaagatggtgatgggatttc

SEQ ID NO: 9 - DNA, 20 bp, Artificial Sequence
  Tata box binding protein primer for real-time RT-PCR
ggcaccactccactgtatcc

SEQ ID NO: 10 - DNA, 20 bp, Artificial Sequence
  Tata box binding protein primer for real-time RT-PCR
gcacaccattttcccagaac

SEQ ID NO: 11 - DNA, 20 bp, Artificial Sequence
  Vimentin primer for real-time RT-PCR
cgaaaacaccctgcaatctt

SEQ ID NO: 12 - DNA, 20 bp, Artificial Sequence
  Vimentin primer for real-time RT-PCR
ttggcagccacactttcata

SEQ ID NO: 13 - DNA, 20 bp, Artificial Sequence
  Thy-1 primer for real-time RT-PCR
agcatcgctctcctgctaac

SEQ ID NO: 14 - DNA, 20 bp, Artificial Sequence
  Thy-1 primer for real-time RT-PCR
gcacgtgcttctttgtctca

SEQ ID NO: 15 - DNA, 22 bp, Artificial Sequence
  Cytokeratin 19 primer for real-time RT-PCR
gaggtggattccgctccgggca

SEQ ID NO: 16 - DNA, 21 bp, Artificial Sequence
  Cytokeratin 19 primer for real-time RT-PCR
atcttcctgtccctcgagcag

SEQ ID NO: 17 - DNA, 19 bp, Artificial Sequence
  EPSTI1 primer for real-time RT-PCR
ctctactgccaggaaatgc

SEQ ID NO: 18 - DNA, 19 bp, Artificial Sequence
  EPSTI1 primer for real-time RT-PCR
gcctgtagcaggatagctc

SEQ ID NO: 19 - DNA, 19 bp, Artificial Sequence
  Adrenal gland protein primer for real-time RT-PCR
ctttttgcagaggccaata

SEQ ID NO: 20 - DNA, 20 bp, Artificial Sequence
  Adrenal gland protein primer for real-time RT-PCR
gtgcgaccgactggaataac

SEQ ID NO: 21 - DNA, 20 bp, Artificial Sequence
  gene specific primer GSP-1
gtagggattaaaatctaaaa

SEQ ID NO: 22 - DNA, 21 bp, Artificial Sequence
  gene specific primer GSP-2
ggtcaagtgtgtgggcagttg

SEQ ID NO: 23 - DNA, 20 bp, Artificial Sequence
  gene specific primer GSP-3
ccaacagcctccagattgct

SEQ ID NO: 24 - DNA, 21 bp, Artificial Sequence
  gene specific primer GSP-4
cccagctgttaccgctattca

SEQ ID NO: 25 - DNA, 20 bp, Artificial Sequence
  gene specific primer GSP-5
gctgccgtttcagttccagt

SEQ ID NO: 26 - DNA, 20 bp, Artificial Sequence
  gene specific primer GSP-6
ggtgaaccggtttagctctg

SEQ ID NO: 27 - DNA, 20 bp, Artificial Sequence
  gene specific primer GSP-7
cttccacttctccaggttgg

SEQ ID NO: 28 - DNA, 19 bp, Artificial Sequence
  gene specific primer GSP-8
ttaggggctgcctccaaac

SEQ ID NO: 29 - DNA, 20 bp, Artificial Sequence
  human specific EPSTI1 primer
caggagtgactggcttctcc

SEQ ID NO: 30 - DNA, 20 bp, Artificial Sequence
  human specific EPSTI1 primer
aagacccccaaagctttcaa

SEQ ID NO: 31 - DNA, 21 bp, Artificial Sequence
  EPSTI1 primer for RT-PCR across the entire ORF
atgaacacccgcaatagagtg

SEQ ID NO: 32 - DNA, 32 bp, Artificial Sequence
  Forward primer for FLAG tagging of EPSTI1
cggtcgacgccaccatgaacacccgcaataga

SEQ ID NO: 33 - DNA, 55 bp, Artificial Sequence
  Reverse primer for FLAG tagging of EPSTI1
ccatcgatggtcacttgtcatcgtcgtccttgtagtctataccccagctgttacc